(19) United States
(12) Patent Application Publication
(10) Pub. No.: US 2001/0046987 A1
(43) Pub. Date: Nov. 29, 2001

(54) Oxazolidinones having a sulfoximine functionality

(76) Inventors: Jackson B. Hester JR., Galesburg, MI (US); David L. Alexander, Kalamazoo, MI (US)

Correspondence Address: Lucy X. Yang, Pharmacia & Upjohn Company, Global Intellectual Property, 301 Henrietta Street, Kalamazoo, MI, 49001, US

(21) Appl. No.: 09/736,858

(22) Filed: Dec. 14, 2000

Related U.S. Application Data
(60) Provisional application No. 60/171,916, filed on Dec. 21, 1999.

(51) Int. Cl. A61K 31/554; A61K 31/42; A61K 31/422; A61K 31/541; C07D 263/04

(52) U.S. Cl. 514/211.15; 514/376; 514/378; 514/227.8; 548/240; 548/225; 544/60; 540/544

(57) ABSTRACT

The present invention provides a compound of formula I

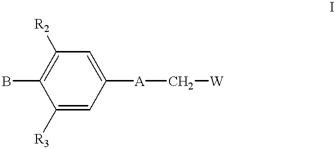

which have potent activities against Gram-positive and Gram-negative bacteria.

### CROSS REFERENCE

This application claims the benefit of the following provisional application: U.S. Ser. No. 60/171,916, filed Dec. 21, 1999, under 35 USC 119(e)(i).

### FIELD OF THE INVENTION

The present invention relates to novel oxazolidinones which have a sulfoximine functionality and their preparations. These compounds have potent activities against Gram-positive and Gram-negative bacteria.

### BACKGROUND OF THE INVENTION

The oxazolidinone antibacterial agents are a novel synthetic class of antimicrobials with potent activity against a number of human and veterinary pathogens, including Gram-positive aerobic bacteria such as multiply-resistant staphylococci and streptococci, anaerobic organisms such as bacteroides and clostridia species, and acid-fast organisms such as Mycobacterium tuberculosis and Mycobacterium avium.

However, oxazolidinones generally do not demostrate an activity at a useful level against aerobic Gram-negative organisms. Thus, the use of these oxazolidinone antibacterial agents is limited to infectious states due to Gram-positive bacteria. Accordingly, it is among the objects of the present invention to provide pharmaceutical compounds which have broader antibacterial activity including the activity against aerobic Gram-negative organisms. We have now discovered that the oxazolidinones of the present invention increase the spectrum of activity to include gram-negative organisms such as Haemophilus influenza and Moraxella catarrhalis.

### INFORMATION DISCLOSURE

U.S. Pat. No. 5,688,792 discloses substituted oxazine and thiazine oxazolidinone useful as antibacticals.

PCT International Publication WO 98/54161 discloses oxazolidinone antibacterial agents having a thiocarbonyl functionality.

U.S. Pat. No. 5,968,962 and PCT International Publication WO 99/29688 discloses phenyloxazolidinones having a C—C bond to 4-8 membered heterocyclic rings.

U.S. Pat. No. 5,952,324 discloses bicyclic oxazine and thiazine oxazolidinone useful as antibacticals.

PCT publications, WO 99/64416, WO99/64417, and WO 00/21960 disclose oxazolidinone derivatives useful as antibacterial agents.

PCT publication, WO 00/10566 discloses isoxazolinones useful as antibacterial agents.

### SUMMARY OF THE INVENTION

The present invention provides a compound of formula I

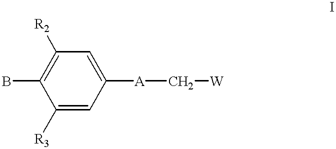

or a pharmaceutically acceptable salt thereof wherein:

A is a structure i, ii, iii, or iv

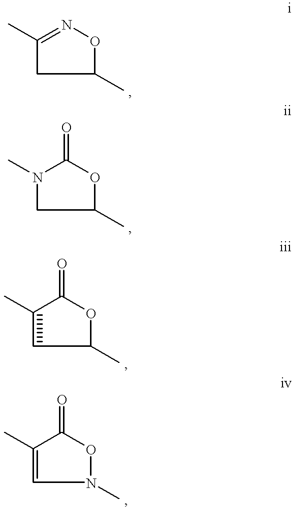

B is

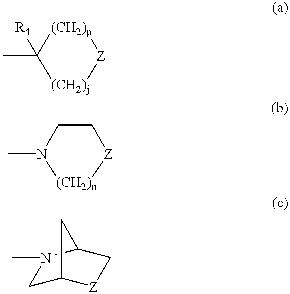

W is NHC(═X)R₁, or —Y—het; povided that when A is a structure iv, W is not —Y—het;

X is O, or S; provided that when X is O, B is not the subsection (b).

Y is NH, O, or S;

Z is S(═O)(═N—R₅);

R₁ is

(a) H,

(b) NH₂,

(c) NHC₁₋₄alkyl,

(d) C₁₋₄alkyl,

(e) C₂₋₄alkenyl,

(f) OC₁₋₄alkyl,

(g) SC₁₋₄alkyl, or

(h) (CH₂)ₚC₃₋₆cycloalkyl;

at each occurrence, alkyl or cycloalkyl in R₁ is optionally substituted with one or more F, Cl or CN;

R₂ and R₃ are independently H, F, Cl, methyl or ethyl;

R₄ is H, CH₃, or F;

R₅ is

(a) H,

(b) C₁₋₄alkyl,

(c) C(═O)C₁₋₄alkyl,

(d) C(═O)OC₁₋₄alkyl,

(e) C(═O)NHR₆, or

(f) C(═S)NHR₆;

R₆ is H, C₁₋₄alkyl, or phenyl;

at each occurrence, alkyl in R₅ and R₆ is optionally substituted with one or more halo, CN, NO₂, phenyl, C₃₋₆cycloalkyl, OR₇, C(═O)R⁷, OC(═O)R₇, C(═O)OR₇, S(═O)ₘR₇, S(═O)ₘNR₇R₇, NR₇SO₂R₇, NR₇SO₂NR₇R₇, NR₇C(═O)R₇, C(═O)NR₇R₇, NR₇R₇, oxo, or oxime;

R₇ is H, C₁₋₄alkyl, or phenyl;

at each occurrence, phenyl is optionally substituted with one or more halo, CN, NO₂, phenyl, C₃₋₆cycloalkyl, OR₇, C(═O)R⁷, OC(═O)R₇, C(═O)OR₇, S(═O)ₘR₇, S(═O)ₘNR₇R₇, NR₇SO₂R₇, NR₇SO₂NR₇R₇, NR₇C(═O)R₇, C(═O)NR₇R₇, or NR₇R₇;

het is a C-linked five-(5) membered heteroaryl ring having 1-4 heteroatoms selected from the group consisting of oxygen, sulfur, and nitrogen, or het is a C-linked six (6) membered heteroaryl ring having 1-3 nitrogen atoms;

p is 0, 1, or 2;

j is 1, 2, 3, 4, or 5; provided that p and j taken together are 2, 3, 4 or 5;

m is 0, 1, or 2;

n is 2 or 3; and [symbol] in structure iii is either a double bond or a single bond.

In another aspect, the present invention also provides:

a pharmaceutical composition comprising a compound of formula I or a pharmaceutically acceptable salt thereof, and a pharmaceutically acceptable carrier,

a method for treating gram-positive microbial infections in humans or other warm-blooded animals by administering to the subject in need a therapeutically effective amount of a compound of formula I or a pharmaceutically acceptable salt thereof, and

a method for treating gram-negative microbial infections in humans or other warm-blooded animals by administering to the subject in need a therapeutically effective amount of a compound of formula I or a pharmaceutically acceptable salt thereof.

The invention also provides some novel intermediates and processes that are useful for preparing compounds of formula I.

### DETAILED DESCRIPTION OF THE INVENTION

The following definitions are used, unless otherwise described.

The term alkyl, alkenyl, etc. refer to both straight and branched groups, but reference to an individual radical such as “propyl” embraces only the straight chain radical, a branched chain isomer such as “isopropyl” being specifically referred to.

The carbon atom content of various hydrocarbon-containing moieties is indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, C₁₋₇ alkyl refers to alkyl of one to seven carbon atoms, inclusive.

The term “halo” refers to fluoro (F), chloro (Cl), bromo (Br), or iodo (I).

The term “het” is a C-linked five-(5) membered heteroaryl ring having 1-4 heteroatoms selected from the group consisting of oxygen, sulfur, and nitrogen, or het is a C-linked six (6) membered heteroaryl ring having 1-3 nitrogen atoms.

Examples of “het” include pyridine, thiophene, furan, pyrazole, pyrimidine, 2-pyridyl, 3-pyridyl, 4-pyridyl, 2-pyrimidinyl, 4-pyrimidinyl, 5-pyrimidinyl, 3-pyridazinyl, 4-pyridazinyl, 3-pyrazinyl, 4-oxo-2-imidazolyl, 2-imidazolyl, 4-imidazolyl, 3-isoxazolyl, 4-isoxazolyl, 5-isoxazolyl, 3-pyrazolyl, 4-pyrazolyl, 5-pyrazolyl, 2-oxazolyl, 4-oxazolyl, 4-oxo-2-oxazolyl, 5-oxazolyl, 1,2,3-oxathiazole, 1,2,3-oxadiazole, 1,2,4-oxadiazole, 1,2,5-oxadiazole, 1,3,4-oxadiazole, 2-thiazolyl, 4-thiazolyl, 5-thiazolyl, 3-isothiazole, 4-isothiazole, 5-isothiazole, 2-furanyl, 3-furanyl, 2-thienyl, 3-thienyl, 2-pyrrolyl, 3-pyrrolyl, 3-isopyrrolyl, 4-isopyrrolyl, 5-isopyrrolyl, 1,2,3,-oxathiazole-1-oxide, 1,2,4-oxadiazol-3-yl, 1,2,4-oxadiazol-5-yl, 5-oxo-1,2,4-oxadiazol-3-yl, 1,2,4-thiadiazol-3-yl, 1,2,4-thiadiazol-5-yl, 3-oxo-1,2,4-thiadiazol-5-yl, 1,3,4-thiadiazol-5-yl, 2-oxo-1,3,4-thiadiazol-5-yl, 1,2,4-triazol-3-yl, 1,2,4-triazol-5-yl, 1,2,3,4-tetrazol-5-yl, 5-oxazolyl, 3-isothiazolyl, 4-isothiazolyl and 5-isothiazolyl, 1,3,4,-oxadiazole, 4-oxo-2-thiazolinyl, or 5-methyl-1,3,4-thiadiazol-2-yl, thiazoledione, 1,2,3,4-thiatriazole, or 1,2,4-dithiazolone.

Mammal refers to human or animals.

The compounds of the present invention are generally named according to the IUPAC or CAS nomenclature system. Abbreviations which are well known to one of ordinary skill in the art may be used (e.g. “Ph” for phenyl, “Me” for methyl, “Et” for ethyl, “h” for hour or hours and “rt” for room temperature).

Specific and preferred values listed below for radicals, substituents, and ranges, are for illustration only; they do not exclude other defined values or other values within defined ranges for the radicals and substituents.

Specifically, alkyl denotes both straight and branched groups; but reference to an individual radical such as “propyl” embraces only the straight chain radical, a branched chain isomer such as “isopropyl” being specifically referred to. Specifically, C₁₋₄alkyl can be methyl, ethyl, propyl, isopropyl, butyl, iso-butyl, sec-butyl, and their isomeric forms thereof.

Specifically, C₂₋₄alkenyl can be vinyl, propenyl, allyl, butenyl, and their isomeric forms thereof; C₃₋₆cycloalkyl can cyclopropyl, cyclobutyl, cyclopentyl, cyclohexyl, and their isomeric forms thereof.

A specific value for A is structure ii as defined above.

A specific value for X is sulfur atom.

A specific value for X is oxygen atom.

A specific value for R₁ is C₁₋₄alkyl.

A more specific value for R₁ is methyl or ethyl.

A specific value for R₁ is cyclopropyl.

A specific value for R₁ is NH₂.

A specific value for R₂ and R₃ are independently H or F.

A specific value for R₂ and R₃ are that one of them is H, the other one is F.

A specific value for R₄ is H or CH₃.

A specific value for R₅ is H.

A specific value for R₅ is C₁₋₄alkyl, optionally substituted with OH.

A specific value for R₅ is CH₃, or ethyl.

A specific value for R₅ is C₁₋₄alkyl substituted with C(═O)NHC₁₋₄alkyl, or C(═O)NH₂.

A specific value for R₅ is C₁₋₄alkyl substituted with phenyl wherein the phenyl is optionally substituted with OH, methyl, NO₂, CF₃, or CN.

A specific value for R₅ is C₁₋₄alkyl substituted with phenyl wherein the phenyl is optionally substituted with NO₂.

A specific value for R₅ is C(═O)NH₂, or C(═O)NHC₁₋₄alkyl.

A specific value for R₅ is C(═O)NHCH₃, or C(═O)NHCH₂CH₃.

A specific value for R₅ is C(═O)C₁₋₄alkyl.

A specific value for R₅ is C(═O)CH₃.

A specific value for R₅ is C(═O)OC₁₋₄alkyl.

A specific value for R₅ is C(═O)OCH₃.

A specific value for het is isoxazol-3-yl, isoxazol-5-yl, 1,2,4-oxadiazol-3-yl, isothiazol-3-yl, 1,2,4-thiadiazol-3-yl or 1,2,5-thiadiazol-3-yl.

The preferred compounds of the present invention are those wherein structure i, ii, or iii has an optical configuration below:

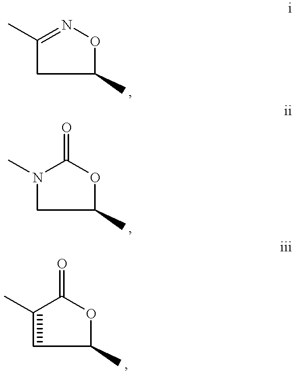

More preferred compounds of the present invention are the compounds of formula IA:

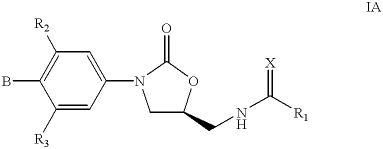

These absolute configurations are called (S)-configuration according to the Cahn-Ingold-Prelog nomenclature system. It will be appreciated by those skilled in the art that compounds of the present invention may have additional chiral centers and be isolated in optically active and racemic forms. The present invention encompasses any racemic, optically-active, tautomeric, or stereoisomeric form, or mixture thereof, of a compound of the invention.

Examples of the present invention are:

(1) N-({(5S)-3-[3-Fluoro-4-(1-imino-1-oxido-1λ⁴,4-thiazinan-4-yl)phenyl]-2-oxo-1,3-oxazolidin-5-yl}methyl)ethanethioamide;

(2) N-({(5S)-3-[3-Fluoro-4-(1-imino-1-oxido-1λ⁴,4-thiazinan-4-yl)phenyl]-2-oxo-1,3-oxazolidin-5-yl}methyl)propanethioamide;

(3) N-({(5S)-3-[3-Fluoro-4-(1-imino-1-oxido-1λ⁴,4-thiazinan-4-yl)phenyl]-2-oxo-1,3-oxazolidin-5-yl}methyl)cyclopropanecarbothioamide;

(4) N-({(5S)-3-[3-Fluoro-4-(1-imino-1-oxidohexahydro-1λ⁴-thiopyran-4-yl)phenyl]-2-oxo-1,3-oxazolidin-5-yl}methyl)acetamide (E)-isomer;

(5) N-({(5S)-3-[3-Fluoro-4-(1-imino-1-oxidohexahydro-1λ⁴-thiopyran-4-yl)phenyl]-2-oxo-1,3-oxazolidin-5-yl}methyl)ethanethioamide (E)-isomer;

(6) N-({(5S)-3-[3-Fluoro-4-(1-imino-1-oxidohexahydro-1λ⁴-thiopyran-4-yl)phenyl]-2-oxo-1,3-oxazolidin-5-yl}methyl)propanethioamide (E)-isomer;

(7) N-({(5S)-3-[3-Fluoro-4-(1-imino-1-oxidohexahydro-1λ⁴-thiopyran-4-yl)phenyl]-2-oxo-1,3-oxazolidin-5-yl}methyl)cyclopropanecarbothioamide (E)-isomer;

(8) N-({(5S)-3-[3-Fluoro-4-(1-imino-1-oxidohexahydro-1λ⁴-thiopyran-4-yl)phenyl]-2-oxo-1,3-oxazolidin-5-yl}methyl)acetamide (Z)-isomer;

(9) N-({(5S)-3-[3-Fluoro-4-(1-imino-1-oxidohexahydro-1λ⁴-thiopyran-4-yl)phenyl]-2-oxo-1,3-oxazolidin-5-yl}methyl)ethanethioamide (Z)-isomer;

(10) N-({(5S)-3-[3-Fluoro-4-(1-imino-1-oxidohexahydro-1λ⁴-thiopyran-4-yl)phenyl]-2-oxo-1,3-oxazolidin-5-yl}methyl)propanethioamide (Z)-isomer;

(11) N-({(5S)-3-[3-Fluoro-4-(1-imino-1-oxidohexahydro-1λ⁴-thiopyran-4-yl)phenyl]-2-oxo-1,3-oxazolidin-5-yl}methyl)cyclopropanethioamide (Z)-isomer;

(12) N-({(5S)-3-[3-Fluoro-4-[1-(acetylimino)-1-oxidohexahydro-1λ⁴-thiopyran-4-yl]phenyl]-2-oxo-1,3-oxazolidin-5-yl}methyl)acetamide, (Z)-isomer;

(13) N-({(5S)-3-[3-Fluoro-4-[1-(methylimino)-1-oxidohexahydro-1λ⁴-thiopyran-4-yl]phenyl]-2-oxo-1,3-oxazolidin-5-yl}methyl)propanethioamide, Z-isomer;

(14) N-({(5S)-3-[3-Fluoro-4-[1-(acetylimino)-1-oxidohexahydro-1λ⁴-thiopyran-4-yl]phenyl]-2-oxo-1,3-oxazolidin-5-yl}methyl)propanethioamide, Z-isomer;

(15) N-({(5S)-3-[3-Fluoro-4-[1-(ethylimino)-1-oxidohexahydro-1λ⁴-thiopyran-4-yl]phenyl]-2-oxo-1,3-oxazolidin-5-yl}methyl)propanethioamide, Z-isomer;

(16) N-({(5S)-3-[3-Fluoro-4-[1-[(phenylmethyl)imino]-1-oxidohexahydro-1λ⁴-thiopyran-4-yl]phenyl]-2-oxo-1,3-oxazolidin-5-yl}methyl)propanethioamide, Z-isomer;

(17) N-({(5S)-3-[3-Fluoro-4-[1-[(3-phenylpropyl)imino]-1-oxidohexahydro-1λ⁴-thiopyran-4-yl]phenyl]-2-oxo-1,3-oxazolidin-5-yl}methyl)propanethioamide, Z-isomer;

(18) N-({(5S)-3-[3-Fluoro-4-(1-{[(methylamino)carbonyl]imino}-1-oxidohexahydro-1λ⁴-thiopyran-4-yl)phenyl]-2-oxo-1,3-oxazolidin-5-yl}methyl)propanethioamide, Z-isomer;

(19) N-({(5S)-3-[3-Fluoro-4-(1-[(methoxycarbonyl)imino]-1-oxidohexahydro-1λ⁴-thiopyran-4-yl)phenyl]-2-oxo-1,3-oxazolidin-5-yl}methyl)propanethioamide, Z-isomer;

(20) N-({(5S)-3-[3-Fluoro-4-(1-[[(ethoxycarbonyl)methyl]imino]-1-oxidohexahydro-1λ⁴-thiopyran-4-yl)phenyl]-2-oxo-1,3-oxazolidin-5-yl}methyl)propanethioamide, Z-isomer;

(21) N-({(5S)-3-[3-Fluoro-4-(1-{[[(4-nitrophenyl)amino]carbonyl]imino}-1-oxidohexahydro-1λ⁴-thiopyran-4-yl)phenyl]-2-oxo-1,3-oxazolidin-5-yl}methyl)propanethioamide, Z-isomer;

(22) N-({(5S)-3-[3-Fluoro-4-[1-[(aminocarbonyl)imino]-1-oxidohexahydro-1λ⁴-thiopyran-4-yl]phenyl]-2-oxo-1,3-oxazolidin-5-yl}methyl)propanethioamide, Z-isomer;

(23) N-({(5S)-3-[3-Fluoro-4-[1-[[(aminocarbonyl)methyl]imino]-1-oxidohexahydro-1λ⁴-thiopyran-4-yl]phenyl]-2-oxo-1,3-oxazolidin-5-yl}methyl)propanethioamide, Z-isomer;

(24) N-({(5S)-3-[3-Fluoro-4-[1-[(2-hydroxyethyl)imino]-1-oxidohexahydro-1λ⁴-thiopyran4-yl]phenyl]-2-oxo-1,3-oxazolidin-5-yl}methyl)propanethioamide, Z-isomer;

(25) N-[((5S)-3-{3-Fluoro-4-[1-(methylimino)-1-oxido-1λ⁴,4-thiazinan-4-yl]phenyl}-2-oxo-1,3-oxazolidin-5-yl)methyl]propanethioamide;

(26) N-[((5S)-3-{3-Fluoro-4-[1-(methylimino)-1-oxido-1λ⁴,4-thiazinan-4-yl]phenyl}-2-oxo-1,3-oxazolidin-5-yl)methyl]cyclopropanecarbothioamide;

(27) N-[((5S)-3-{3-Fluoro-4-(1-[(methoxycarbonyl)imino]-1-oxido-1λ⁴,4-thiazinan-4-yl)phenyl}-2-oxo-1,3-oxazolidin-5-yl)methyl]propanethioamide;

(28) N-[((5S)-3-{3-Fluoro-4-(1-[(methoxycarbonyl)imino]-1-oxido-1λ⁴,4-thiazinan-4-yl)phenyl}-2-oxo-1,3-oxazolidin-5-yl)methyl]cyclopropanecarbothioamide;

(29) N-({(5S)-3-[3-Fluoro-4-[1-(methylimino)-1-oxidohexahydro-1λ⁴-thiopyran-4-yl]phenyl]-2-oxo-1,3-oxazolidin-5-yl}methyl)cyclopropanecarbothioamide, Z-isomer;

(30) N-[((5S)-3-{3-Fluoro-4-[1-[(methoxycarbonyl)imino]-1-oxidohexahydro-1λ⁴-thiopyran-4-yl]phenyl}-2-oxo-1,3-oxazolidin-5-yl)methyl]cyclopropanecarbothioamide, Z-isomer;

(31) N-[((5S)-3-{3-Fluoro-4-[1-(methylimino)-1-oxidohexahydro-1λ⁴-thiopyran-4-yl]phenyl}-2-oxo-1,3-oxazolidin-5-yl)methyl]cyclopropanecarbothioamide, E-isomer;

(32) N-[((5S)-3-{3-Fluoro-4-[1-(methylimino)-1-oxidohexahydro-1λ⁴-thiopyran-4-yl]phenyl}-2-oxo-1,3-oxazolidin-5-yl)methyl]propanethioamide, E-isomer;

(33) N-[((5S)-3-{3-Fluoro-4-[1-[[(phenylmethoxy)carbnonyl]imino]-1-oxidohexahydro-1λ⁴-thiopyran-4-yl]phenyl}-2-oxo-1,3-oxazolidin-5-yl)methyl]acetamide, Z-isomer; or

(34) N-({(5S)-3-[3-Fluoro-4-(1-{[(benzylamino)carbonyl]imino}-1-oxidohexahydro-1λ⁴-thiopyran-4-yl)phenyl]-2-oxo-1,3-oxazolidin-5-yl}methyl)acetamide, Z-isomer.

The following Schemes describe the preparation of compounds of the present invention. All of the starting materials are prepared by procedures described in these schemes or by procedures that would be well known to one of ordinary skill in organic chemistry. The variables used in the Schemes are as defined below or as in the claims.

The compounds of this invention can be prepared in accordance to one or more of the Schemes discussed below. Optically pure material could be obtained either by one of a number of asymmetric syntheses or alternatively by resolution from a racemic mixture.

In scheme I the starting materials of 1-a can be prepared according to the procedures described in U.S. Pat. No. 5,688,792 or PCT International Publication WO 98/54161. A compound of 1-a is allowed to react with sodium azide in polyphosphoric acid at a temperature in a range from about 40° C. to about 70° C. to provide compound 1-b. Compound 1-b can be alkylated by the reaction with aldehydes or ketones and formic acid using Leuckart-Wallach or Eschweiler-Clarke reaction conditions to provide 1-c (R′═C₁₋₄alkyl). An illustration of this method for the methylation of compound 1-b is described in Preparation 3 of the present invention. In an alternative method for this alkylation, a compound of 1-b or 1-e (R′═H) is allowed to react with an aldehyde or ketone, triethylsilane and trifluoroacetic acid. Para-formaldehyde is a convenient source of formaldehyde for this reaction and aldehydes protected as acetals can also be employed. Solvents such as toluene, dichloromethane, THF, and preferably acetonitrile with temperatures, depending on the solvent, in the range of 10°-120° C. can be used. This method is illustrated in Examples 13 and 16. Aldehydes with various functional groups can also be employed in this reaction as illustrated by the use of ethyl glyoxalate in Example 20. The ester prepared in this example can be reduced to an alcohol with lithium borohydride (Example 24) or converted to an amide with ammonium hydroxide (Example 23). Compounds wherein R₅ is C₁₋₄alkyl substituted by NH₂ or NHalkyl can be obtained by employing an amine protecting group such as benzyloxycarbonyl or tert-butyloxycarbonyl that can subsequently be removed. Other compounds wherein R₅ is substituted alkyl can be obtained with appropriate modifications of this reductive alkylation procedure.

The acetamide 1-c is hydrolyzed to the corresponding amine, 1-d, with hydrochloric acid in a solvent such as methanol at the reflux temperature. Acylation of the amine with appropriate dithioesters and a tertiary amine base such as triethylamine provides the corresponding compound 1-e. Solvents such as CH₂Cl₂, THF or preferably MeOH and temperatures of 24° C. to the reflux temperature of the solvent are suitable for this reaction. Preparations of other thiocarbonyl compounds 1-e are as described in PCT International Publication WO 98/54161. Where R′ is hydrogen, 1-e may be converted to compound 1-f with additional functional groups on the sulfoximine nitrogen. Reactions with carboxylic acid chlorides or anhydrides in solvents such as pyridine at a temperature in a range from about 24-100° C. provide the corresponding acyl derivatives (R₅ is C(═O)C₁₋₄alkyl). Carboxylic acid anhydrides in the corresponding carboxylic acid as solvent can also be used as illustrated for acetic anhydride in acetic acid in Preparation 2. Carbamates (R₅ is C(═O)OC₁₋₄alkyl) are prepared by the reactions of 1-e (R′ is H) with appropriate alkyl chloroformates in pyridine at 0° C. to 100° C. In addition, 4-(dimethylamino)pyridine can be used to catalyze this reaction as illustrated in Example 19. Alkyl ureas and alkyl thioureas (R₆ is C₁₋₄alkyl) are prepared by warming 1-e (R′ is H) with the appropriate alkyl isocyanate or alkyl isothiocyanate at a temperature in a range from about 30° C. to about 100° C. DMF is a preferred solvent for this reaction. Compounds where R₆ is phenyl or substituted phenyl are similarly preprared. Compounds where R₆ is hydrogen are prepared by the reactions of 1-e (R′ is H) with sodium cyanate or sodium thiocyanate in acetic acid at a temperature in a range from about 24° C. to about 100° C. For the preparation of a compound wherein X is oxygen, the amine 1-d may be acylated with appropriate carbonyl derivatives such as carboxylic acid anhydrides, alkyl chloroformates, alkyl isocyanates and sodium cyanate in an acetic acid solution. Compounds of formula I wherein B is the subsection (c) can be prepared by the methods shown in Scheme I with the starting material, sulfoxides. The sulfoxides can be prepared according to the procedure disclosed in U.S. Pat. No. 5,952,324.

Scheme II illustrates the preparation of compounds of 2-e and 2-f. The starting material 2-a can be prepared according to the procedures described in U.S. Pat. No. 5,968,962, PCT International Publication WO 99/29688 and PCT International Publication WO 98/54161. In this series the sulfoxides can be either cis or trans to the benzene ring attachment. The reaction of compounds 2-a with O-mesitylenesulfonylhydroxylamine (MSH) proceeds with retention of the sulfoxide stereochemistry in the products 2-b. This reaction is usually carried out at ambient temperature in solvents such as methylene chloride. Subsequent reactions in Scheme II are carried out as discussed for the corresponding steps in Scheme I. Compounds 2-e where X═O are prepared by acylating compounds 2-d with appropriate carbonyl derivatives such as carboxylic acid anhydrides, alkyl chloroformates, alkyl isocyanates and sodium cyanate in acetic acid.

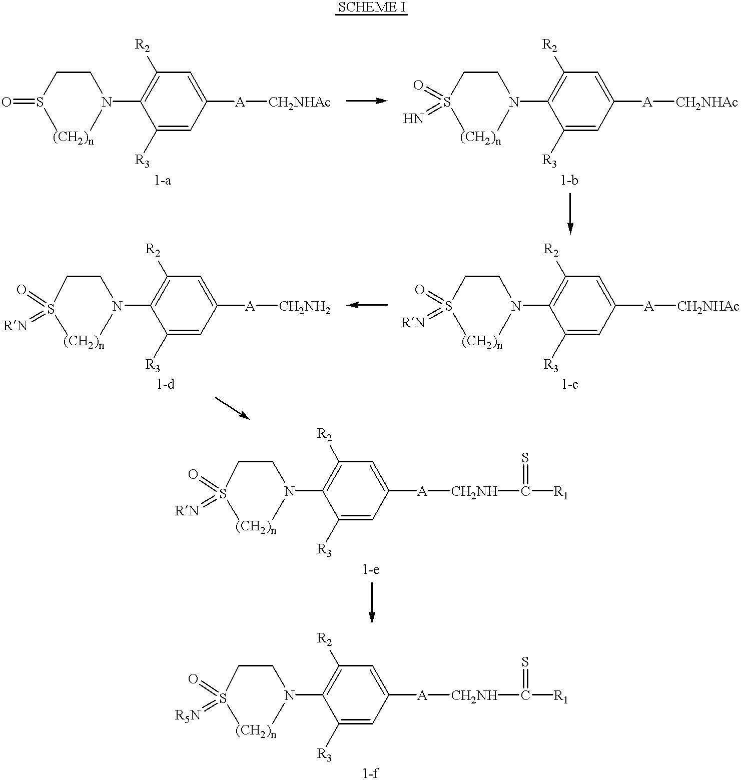

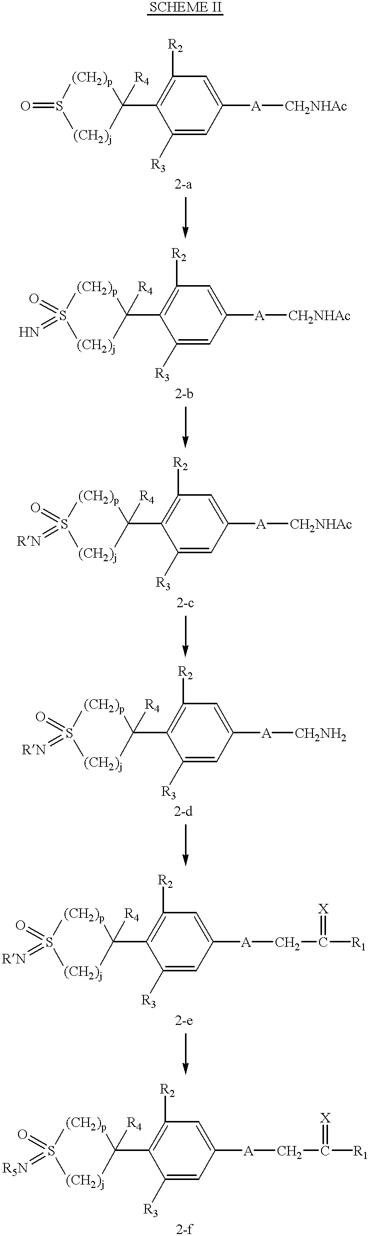

The pharmaceutical compositions of this invention may be prepared by combining the compounds of formula I of this invention with a solid or liquid pharmaceutically acceptable carrier and, optionally, with pharmaceutically acceptable adjuvants and excipient employing standard and conventional techniques. Solid form compositions include powders, tablets, dispersible granules, capsules, cachets and suppositories. A solid carrier can be at least one substance which may also function as a diluent, flavoring agent, solubilizer, lubricant, suspending agent, binder, tablet disintegrating agent, and encapsulating agent. Inert solid carriers include magnesium carbonate, magnesium stearate, talc, sugar, lactose, pectin, dextrin, starch, gelatin, cellulosic materials, low melting wax, cocoa butter, and the like. Liquid form compositions include solutions, suspensions and emulsions. For example, there may be provided solutions of the compounds of this invention dissolved in water and water-propylene glycol and water-polyethylene glycol systems, optionally containing suitable conventional coloring agents, flavoring agents, stabilizers and thickening agents.

Preferably, the pharmaceutical composition is provided employing conventional techniques in unit dosage form containing effective or appropriate amounts of the active component, that is, the compounds of formula I according to this invention.

The quantity of active component, that is the compound of formula I according to this invention, in the pharmaceutical composition and unit dosage form thereof may be varied or adjusted widely depending upon the particular application, the potency of the particular compound and the desired concentration. Generally, the quantity of active component will range between 0.5% to 90% by weight of the composition.

In therapeutic use for treating, or combating, bacterial infections in warm-blooded animals, the compounds or pharmaceutical compositions thereof will be administered orally, topically, transdermally, and/or parenterally at a dosage to obtain and maintain a concentration, that is, an amount, or blood-level of active component in the animal undergoing treatment which will be antibacterially effective. Generally, such antibacterially effective amount of dosage of active component will be in the range of about 0.1 to about 100, more preferably about 1.0 to about 50 mg/kg of body weight/day. It is to be understood that the dosages may vary depending upon the requirements of the patient, the severity of the bacterial infection being treated, and the particular compound being used. Also, it is to be understood that the initial dosage administered may be increased beyond the above upper level in order to rapidly achieve the desired blood-level or the initial dosage may be smaller than the optimum and the daily dosage may be progressively increased during the course of treatment depending on the particular situation. If desired, the daily dose may also be divided into multiple doses for administration, e.g., two to four times per day.

The compounds of formula I according to this invention are administered parenterally, i.e., by injection, for example, by intravenous injection or by other parenteral routes of administration. Pharmaceutical compositions for parenteral administration will generally contain a pharmaceutically acceptable amount of the compound according to formula I as a soluble salt (acid addition salt or base salt) dissolved in a pharmaceutically acceptable liquid carrier such as, for example, water-for-injection and a buffer to provide a suitably buffered isotonic solution, for example, having a pH of about 3.5-6. Suitable buffering agents include, for example, trisodium orthophosphate, sodium bicarbonate, sodium citrate, N-methylglucamine, L(+)-lysine and L(+)-arginine to name but a few representative buffering agents. The compounds according to formula I generally will be dissolved in the carrier in an amount sufficient to provide a pharmaceutically acceptable injectable concentration in the range of about 1 mg/ml to about 400 mg/ml of solution. The resulting liquid pharmaceutical composition will be administered so as to obtain the above-mentioned antibacterially effective amount of dosage. The compounds of formula I according to this invention are advantageously administered orally in solid and liquid dosage forms.

The oxazolidinone antibacterial agents of this invention have useful activity against a variety of organisms. The in vitro activity of compounds of this invention can be assessed by standard testing procedures such as the determination of minimum inhibitory concentration (MIC) by agar dilution as described in “Approved Standard. Methods for Dilution Antimicrobial Susceptibility Tests for Bacteria That Grow Aerobically”, 3rd. ed., published 1993 by the National Committee for Clinical Laboratory Standards, Villanova, Pa., USA. The activity of compounds of this invention against Staphylococcus aureus, Staphylococcus epidermidis, Enterococcus faecium, Streptococcus pneumoniae, Streptococcus pyogenes, Enterococcus faecalis, Moraxella catarrhalis and H. influenzae is shown in Table 1.

TABLE 1
Antibacterial Activity Minimum Inhibitory Concentration (μg/mL)

| Example | SAUR | SEPI | EFAE | SPNE | SPYO | HINF | EFAE | MCAT |
|---|---|---|---|---|---|---|---|---|
| No. | 9213 | 30593 | 12712 | 9912 | 152 | 30063 | 9217 | 30607 |
| 1 | 1 | 0.5 | 1 | 0.25 | 0.25 | 4 | 1 | 2 |
| 2 | 1 | 0.25 | 0.5 | 0.25 | 0.25 | 2 | 0.5 | 2 |
| 3 | 1 | 0.25 | 0.5 | 0.25 | 0.25 | 4 | 1 | 1 |
| 4 | 16 | 2 | 4 | 1 | 1 | 8 | 2 | 4 |
| 8 | 8 | 1 | 2 | 0.5 | 1 | 8 | 2 | 2 |
| 9 | 0.5 | 0.125 | 0.5 | 0.25 | 0.25 | 2 | 0.5 | 2 |
| 10 | 1 | 0.25 | 0.5 | 0.25 | 0.25 | 2 | 0.5 | 2 |
| 11 | 1 | 0.5 | 0.5 | 0.25 | 0.25 | 4 | 0.5 | 1 |
| 12 | 8 | 1 | | 0.5 | 1 | 16 | 2 | 8 |
| 13 | 1 | 0.5 | 1 | 0.25 | 0.25 | 8 | 0.5 | 2 |
| 14 | 1 | 0.5 | 1 | 0.25 | 0.25 | 4 | 0.5 | 2 |
| 15 | 2 | 1 | 1 | 0.5 | 0.5 | 8 | 1 | 2 |
| 16 | 2 | 1 | 1 | 0.25 | 0.5 | >64 | 1 | 2 |
| 17 | 2 | 2 | 2 | 0.5 | 1 | >64 | 1 | 2 |
| 18 | 2 | 1 | 1 | 0.25 | 0.5 | 8 | 1 | 4 |
| 19 | 1 | 0.5 | 1 | 0.25 | 0.25 | 2 | | 2 |
| 20 | 2 | 1 | 1 | 4 | 16 | 8 | 1 | 4 |
| 21 | 0.5 | 0.25 | 0.25 | <0.06 | 0.125 | >64 | 0.25 | 0.25 |
| 22 | 2 | .05 | 0.5 | 0.125 | 0.5 | 4 | 0.5 | 2 |
| 23 | 2 | 2 | 1 | 0.25 | 0.5 | 4 | 0.5 | 4 |
| 24 | 2 | 0.5 | 1 | 0.25 | 0.5 | 4 | 1 | 4 |
| 25 | 2 | 0.5 | 1 | 0.25 | 0.5 | 4 | 1 | 4 |
| 26 | 2 | 0.5 | 1 | 0.25 | 0.5 | 4 | 0.5 | 2 |
| 27 | 2 | 0.5 | 0.5 | 0.125 | 0.5 | 4 | 0.5 | 8 |
| 28 | 2 | 0.5 | 1 | 0.25 | 0.5 | 4 | 1 | 2 |
| 29 | 1 | 0.5 | 1 | 0.25 | 0.5 | 4 | 0.5 | 2 |
| 30 | 1 | 0.5 | 0.5 | 0.125 | 0.5 | 4 | 0.5 | 0.5 |
| 31 | 2 | 1 | 2 | 0.5 | 1 | 8 | 1 | 4 |
| 32 | 4 | 1 | 2 | 0.5 | 1 | 8 | 2 | 8 |
| 33 | 8 | 2 | 4 | 1 | 1 | 64 | 4 | 16 |
| 34 | 16 | 2 | 4 | 1 | 2 | 32 | 4 | 16 |
| PREP 1 | 16 | 4 | 4 | 1 | 2 | 16 | 8 | 8 |
| PREP 2 | 16 | 2 | 4 | 1 | 2 | 32 | 2 | 8 |
| PREP 3 | 16 | 2 | 4 | 1 | 2 | 16 | 4 | 8 |

### EXAMPLES

### Preparation 1

### N-({(5S)-3-[3-Fluoro-4-(1-imino-1-oxido-1λ⁴,4-thiazinan-4-yl)phenyl]-2-oxo-1,3-oxazolidin-5-yl}methyl)acetamide (2)

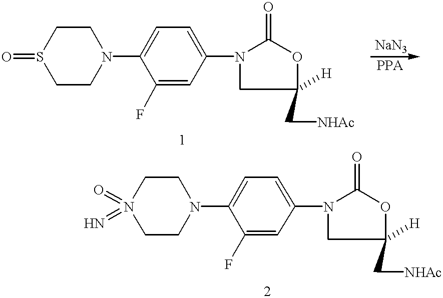

((S)-N-[[3-[3-fluoro-4-(1-oxothiomorpholin-4-yl)phenyl]-2-oxo-5-oxazolidinyl]methyl]acetamide, (compound 1, prepared according to the procedure described in WO95/07271, Example 3) (1.01 g, 2.73 mmol) and sodium azide (0.38 g, 5.8 mmol) are added at ambient temperature, under nitrogen, with stirring to polyphosphoric acid (40 g) and the mixture is warmed at 50-55° C. for 6 hours and at 60° C. for 4 hours, cooled slowly to 0° C. and treated, dropwise with water (20 ml) and enough 50% (w/w) sodium hydroxide to raise the pH to 10.5-11.0. This mixture is diluted with enough water to give a solution which is extracted with CHCl₃. The extract is dried (Na₂SO₄) and concentrated. Chromatography of the residue on silica gel with mixtures of MeOH—CHCl₃ containing 2-3% MeOH gave 691 mg of the product. Crystallization of this material from acetone-hexane gave compound 2.

mp 165-166° C.; HRMS (FAB) calcd for C₁₆H₂₂FN₄O₄S (M+H⁺) 385.1346, found 385.1352. Anal. Calcd for C₁₆H₂₁FN₄O₄S: C, 49.99; H, 5.51; N, 14.57. Found: C, 50.01; H, 5.56; N, 14.49.

### Preparation 2

### N-({(5S)-3-[3-Fluoro-4-(1-acetylimino-1-oxido-1λ⁴,4-thiazinan-4-yl)phenyl]-2-oxo-1,3-oxazolidin-5-yl}methyl)acetamide (7)

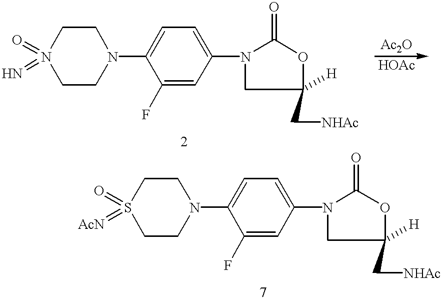

A stirred solution of 2 (100 mg, 0.26 mmol) in acetic acid (1 ml), under nitrogen, is treated with acetic anhydride (55 μL, 0.58 mmol), kept at ambient temperature (24° C.) for 66 hours and concentrated in vacuo. Chromatography of the residue on silica gel with 3% MeOH—CHCl₃ gave the product which is recrystallized from MeOH to give 68 mg of 7.

mp 219.5-221.0° C.; HRMS (FAB) calcd for C₁₈H₂₄FN₄O₅S (M+H⁺) 427.1451, found 427.1458. Anal. Calcd for C₁₈H₂₃FN₄O₅S: C, 50.69; H, 5.44; N, 13.14. Found: 50.64; H, 5.49; N, 13.12.

### Preparation 3

### N-({(5S)-3-[3-Fluoro-4-(1-methylimino-1-oxido-1λ⁴,4-thiazinan-4-yl)phenyl]-2-oxo-1,3-oxazolidin-5-yl }methyl)acetamide (8)

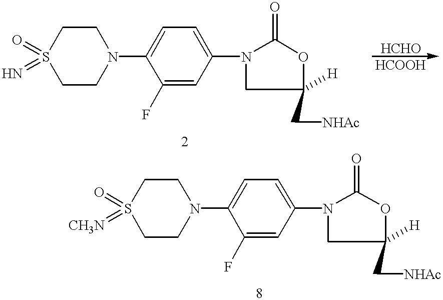

A stirred mixture of 2 (230 mg, 0.60 mmol), 37.5% aqueous formaldehyde (75 μL), 1.0 mmol) and formic acid (75 μL, 2.0 mmol) is warmed at 80° C. for 4 hours, treated with additional formaldehyde (75 μL) and formic acid (75 μL) and warmed at 80° C. for an additional 4 hours. The cooled mixture is dissolved in CHCl₃ and water and treated with 1 N NaOH to pH 10. It is extracted with CHCl₃ and the extract is dried (Na₂SO₄) and concentrated. The residue is combined with the crude product from a similar reaction with 53 mg of 2 and chromatographed on silica gel with mixtures of MeOH—CHCl₃ containing 2-4% MeOH to give 140 mg of 8.

HRMS (ESI) calcd for C₁₇H₂₄FN₄O₄S (M+H⁺) 399.1502, found 399.1498.

### Example 1

### N-({(5S)-3-[3-Fluoro-4-(1-imino-1-oxido-1λ⁴,4-thiazinan-4-yl)phenyl]-2-oxo-1,3-oxazolidin-5-yl}methyl)ethanethioamide (4)

Step 1:

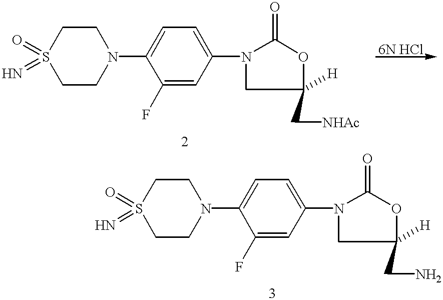

A stirred mixture of 2 (691 mg, 1.80 mmol), MeOH (30 ml) and 6 N hydrochloric acid (10 mL) is gently refluxed for 21 hours, cooled and neutralized (pH7) with 1 N NaOH. It is concentrated in vacuo and the residue is dissolved in a small amount of water, adjusted to pH 11 with NaOH and extracted with CHCl₃ and 5% MeOH—CH₂Cl₂. The extracts are dried (Na₂SO₄) and concentrated to give 535 mg of 3.

Step 2:

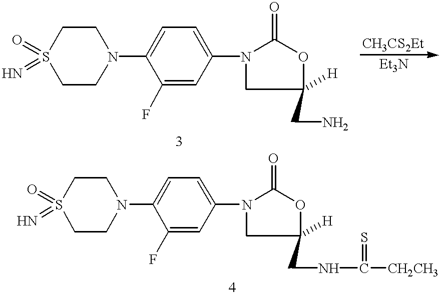

A stirred solution of 3 (371 mg, 1.08 mmol) in MeOH (10 ml) is treated with triethylamine (302 μL, 2.17 mmol) and ethyl dithioacetate (162 μL, 1.41 mmol) and warmed at 40° C., under nitrogen, for 17 hours. The solid product is chromatographed on silica gel with 2% MeOH—CH₂Cl₂ and the resulting product is crystallized from EtOH—CH₃CN to give 298 mg of 4.

mp 197-198° C.; HRMS (FAB) calcd for C₁₆H₂₂FN₄O₃S₂ (M+H⁺) 401.1117, found 401.1115. Anal. Calcd for C₁₆H₂₁FN₄O₃S₂: C, 47.98; H, 5.28; N, 13.99. Found: C, 47.98; H, 5.34; N, 14.01.

### Example 2

### N-({(5S)-3-[3-Fluoro-4-(1-imino-1-oxido-1λ⁴,4-thiazinan-4-yl)phenyl]-2-oxo-1,3-oxazolidin-5-yl}methyl)propanethioamide (5)

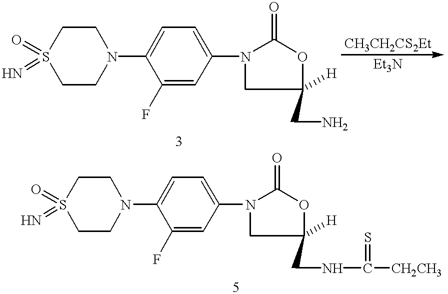

As described in Example 1, Step 2 the reaction of 3 with ethyl dithiopropionate and triethylamine in methanol gave 5 which is crystallized from MeOH.

mp 189-190° C.; HRMS (FAB) calcd for C₁₇H₂₄FN₄O₃S₂ (M+H⁺) 415.1273, found 415.1278. Anal calcd for C₁₇H₂₃FN₄O₃S₂: C, 49.26; H, 5.59; N, 13.52. Found: C, 49.89; H, 5.81; N, 13.18.

### Example 3

### N-({(5S)-3-[3-Fluoro-4-(1-imino-1-oxido-1λ⁴,4-thiazinan-4-yl)phenyl]-2-oxo-1,3-oxazolidin-5-yl}methyl)cyclopropanecarbothioamide (6)

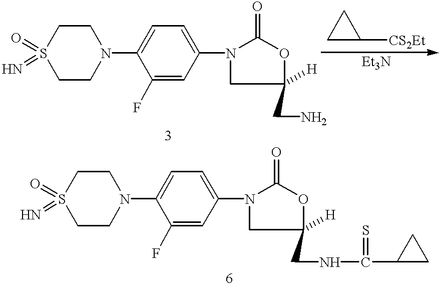

As described in Example 1, Step 2 the reaction of 3 with ethyl dithiocyclopropane-carboxylate and triethylamine in MeOH gave 6 which is crystallized from MeOH.

mp 209-210° C. (dec); HRMS (FAB) calcd for C₁₈H₂₄FN₄O₃S₂ (M+H⁺) 427.1273, found 427.1289. Anal. Calcd for C₁₈H₂₃FN₄O₃S₂: C, 50.69; H, 5.43; N, 13.14. Found: C, 50.70; H, 5.50; N, 13.00.

### Example 4

### N-({(5S)-3-[3-Fluoro-4-(1-imino-1-oxidohexahydro-1λ⁴-thiopyran-4-yl)phenyl]-2-oxo-1,3-oxazolidin-5-yl}methyl)acetamide, E-Isomer (10)

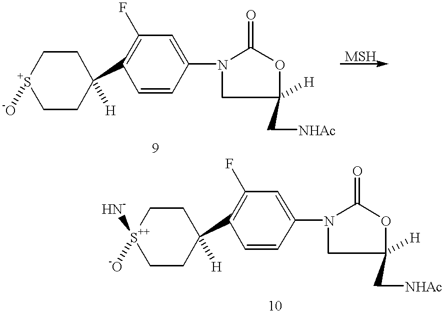

Step 1:

A stirred, ice cold solution of ethyl O-(mesitylenesulfonyl)acetohydroxamate (1.28 g, 4.49 mmol) in dioxane (3 ml), under nitrogen, is treated dropwise during 5 minutes with 70% perchloric acid (0.48 ml, 5.57 mmol) and kept in the ice bath for 4 hours. It is then poured with stirring into ice water (30 ml), stirred for 30 minutes at 0° C. and filtered. The solid is ished with cold water and dissolved in a small amount of diethyl ether. The solution is ished with water, dried (K₂CO₃) and the product (O-mesitylenesulfonyl-hydroxylamine, MSH) is crystallized, under nitrogen from cold Et₂O-pentane. A CH₂Cl₂ solution of this product is used in Step 2.

Step 2:

A stirred solution of 9 ((S)-trans-(−)-N-[[3-[3-fluoro-4-(tetrahydro-1-oxido-2H-thiopyran-4-yl)phenyl]-2-oxo-5-oxazolidinyl]methyl]acetamide (prepared according to the procedure described in WO95/07271, Example 9, Step 1) (470 mg, 1.28 mmol) in CH₂Cl₂ (5 ml) is treated with a CH₂Cl₂ solution of the MSH prepared in Step 1 and kept at ambient temperature (24° C.) for 19 hours. It is mixed with water and 5% MeOH—CH₂Cl₂, treated with 1 N NaOH to pH 11 and extracted with 5% MeOH—CH₂Cl₂. The extract is dried (Na₂SO₄) and concentrated. Chromatography of the residue on silica gel with 2.5% MeOH-0.1% NH₄OH—CH₂Cl₂ gives 10 which can be crystallized from MeOH:

Mp 225-226° C.; HRMS (FAB) calcd for C₁₇H₂₃FN₃O₄S (M+H⁺) 384.1393, found 384.1398. Anal calcd for C₁₇H₂₂FN₃O₄S: C, 53.25; H, 5.78; N, 10.96. Found: C, 53.18; H, 5.90; N, 10.79.

### Example 5

### N-({(5S)-3-[3-Fluoro-4-(1-imino-1-oxidohexahydro-1λ⁴-thiopyran-4-yl)phenyl]-2-oxo-1,3-oxazolidin-5-yl}methyl)ethanethioamide, E-Isomer (12)

Step 1

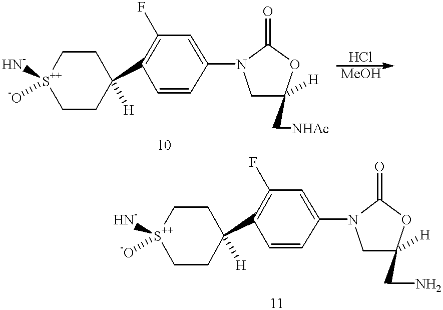

A stirred mixture of 10 (586 mg, 1.53 mmol), MeOH (24 ml) and water (4 ml) is treated with concentrated hydrochloric acid (4 ml), refluxed for 22 hours, neutralized with 50% NaOH and concentrated in vacuo to remove MeOH. The residue is diluted with brine, treated with 1 N NaOH to pH 11 and extracted with 5% MeOH—CH₂Cl₂. The extract is dried (Na₂SO₄) and concentrated to give 464 mg of 11.

Step 2:

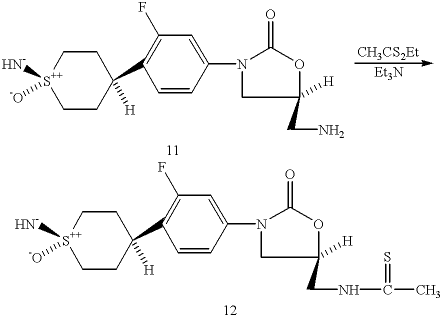

A stirred solution of 11 (159 mg, 0.47 mmol) in MeOH (5 ml) is treated with ethyl dithioacetate (73 μL, 0.64 mmol) and triethylamine (130 μL, 0.93 mmol), kept at about 40° C. for 24 hours, cooled and concentrated under a stream of nitrogen. Chromatography of the residue on silica gel first with 2% MeOH-0.1% Et₃N—CHCl₃, and then with 4% EtOH-0.1% Et₃N—CHCl₃ and crystallization of the product from acetone give 94 mg of the title compound 12.

Mp 193-194° C. (dec); HRMS (FAB) calcd for C₁₇H₂₃FN₃O₃S₂ (M+H⁺) 400.1165, found 400.1157. Anal calcd for C₁₇H₂₂FN₃O₃S₂: C, 51.11; H, 5.55; N, 10.52. Found: C, 51.07; H, 5.61; N, 10.37.

### Example 6

N-({(5S)-3-[3-Fluoro-4-(1-imino-1-oxidohexahydro-1λ⁴-thiopyran-4-yl)phenyl]-2-oxo-1,3-oxazolidin-5-yl}methyl)propanethioamide, E-Isomer (13)

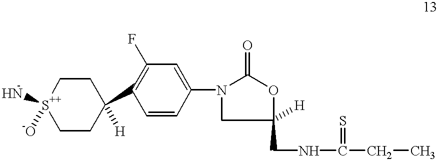

As described in Example 5, Step 2 the reaction of 11 with ethyl dithiopropionate and triethylamine in MeOH at 40° C. gives 13 which is cyrstallized from acetone.

Mp 191-192° C. (dec); HRMS (FAB) calcd for C₁₈H₂₅FN₃O₃S₂: (M+H⁺) 414.1321, found 414.1329. Anal. Caled for C₁₈H₂₄FN₃O₃S₂: C, 52.28; H, 5.85; N, 10.16. Found: C, 52.30; H, 5.90; N, 10.14.

### Example 7

### N-({(5S)-3-[3-Fluoro-4-(1-imino-1-oxidohexahydro-1λ⁴-thiopyran4-yl)phenyl]-2-oxo-1,3-oxazolidin-5-yl}methyl)cyclopropanecarbothioamide, E-Isomer (14)

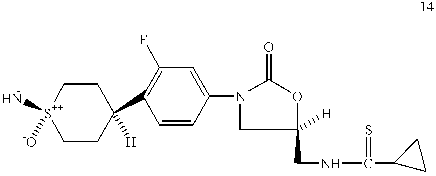

As described in Example 5, Step 2 the reaction of 11 with ethyl dithiocyclopropanecarboxylate and triethylamine in MeOH at 40° C. gives 14 which is crystallized from acetone-MeOH.

Mp. 210-211° C. (dec); HRMS (FAB) calcd for C₁₉H₂₅FN₃O₃S₂: (M+H⁺) 426.1321, found 426.1309. Anal. Calcd for C₁₉H₂₄FN₃O₃S₂: C, 53.63; H, 5.68; N, 9.87. Found: C, 53.68; H, 5.74; N, 9.84.

### Example 8

### N-({(5S)-3-[3-Fluoro-4-(1-imino-1-oxidohexahydro-1λ⁴-thiopyran-4-yl)phenyl]-2-oxo-1,3-oxazolidin-5-yl}methyl)acetamide, Z-Isomer (15)

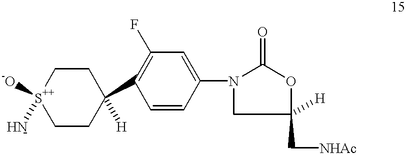

As described in Example 4 the reaction of (S)-cis-(−)-N-[[3-[3-fluoro-4-(tetrahydro-1-oxido-2H-thiopyran-4-yl)phenyl]-2-oxo-5-oxazolidinyl]methyl]acetamide (see WO 98/54161, Example 7, Step 1) with MSH gave 15 which is crystallized from EtOAc.

mp 189.5-190.5° C.; HRMS (FAB) calcd for C₁₇H₂₃FN₃O₄S (M+H⁺) 384.1393, found 384.1389. Anal. Calcd for C₁₇H₂₂FN₃O₄S: C, 53.25; H, 5.78; N, 10.96. Found: C, 53.21; H, 5.82; N, 10.88.

### Example 9

### N-({(5S)-3-[3-Fluoro-4-(1-imino-1-oxidohexahydro-1λ⁴-thiopyran-4-yl)phenyl]-2-oxo-1,3-oxazolidin-5-yl}methyl)ethanethioamide, Z-Isomer (17)

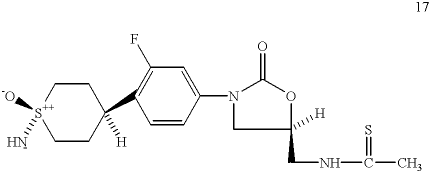

As described in Example 5 compound 15 is hydrolyzed with 6 N hydrochloric acid in methanol and the resulting amine (16) is condensed with ethyl dithioacetate and triethylamine in methanol to give 17 which is crystallized from MeOH.

Mp 206-207° C.; HRMS (FAB) calcd for C₁₇H₂₃FN₃O₃S₂ (M+H⁺) 400.1165, found 400.1171. Anal. Calcd for C₁₇H₂₂FN₃O₃S₂: C, 51.11; H, 5.55; N, 10.52. Found: C, 51.65; H, 5.77; N, 10.28.

### Example 10

### N-({(5S)-3-[3-Fluoro-4-(1-imino-1-oxidohexahydro-1λ⁴-thiopyran-4-yl)phenyl]-2-oxo-1,3-oxazolidin-5-yl}methyl)propanethioamide, Z-Isomer (18)

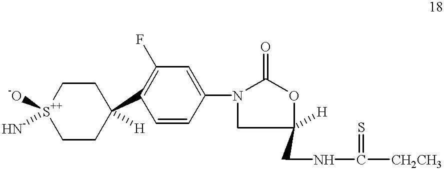

As described in Example 9 the amine (16) is allowed to react with ethyl dithiopropionate and triethylamine in methanol to give 18 which is recrystallized from methanol.

Mp 211-213° C.; HRMS (FAB) calcd for C₁₈H₂₅FN₃O₃S₂ (M+H⁺) 414.1321, found 414.1313. Anal. Calcd for C₁₈H₂₄FN₃O₃S₂: C, 52.28; H, 5.85; H, 10.16. Found: C, 52.33; H, 5.95; H, 10.11.

### Example 11

### N-({(5S)-3-[3-Fluoro-4-(1-imino-1-oxidohexahydro-1λ⁴-thiopyran-4-yl)phenyl]-2-oxo-1,3-oxazolidin-5-yl}methyl)cyclopropanethioamide, Z-Isomer (19)

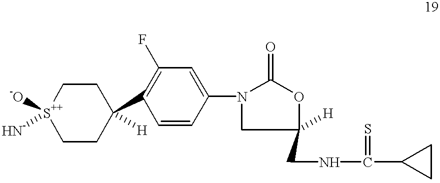

As described in Example 9 the amine (16) is allowed to react with ethyl dithiocyclopropanecarboxylate and triethylamine in methanol to give 19 which is recrystallized from methanol.

Mp 220-221° C.; HRMS (FAB) calcd for C₁₉H₂₅FN₃O₃S₂ (M+H⁺) 426.1321, found 426.1317. Anal. Calcd for C₁₉H₂₄FN₃O₃S₂.0.55 MeOH: C, 52.99; H, 5.96; N, 9.48. Found: C, 52.50; H, 5.80; N, 9.49.

### Example 12

### N-({(5S)-3-[3-Fluoro-4-[1-(acetylimino)-1-oxidohexahydro-1λ⁴-thiopyran-4-yl]phenyl]-2-oxo-1,3-oxazolidin-5-yl}methyl)acetamide, Z-isomer (20)

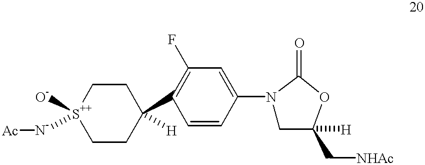

As described in Preparation 2, compound 15 (Example 8) is allowed to react with acetic anhydride in acetic acid to give 20 which is recrystallized from CH₂Cl₂—MeOH.

Mp 237.5-239° C.; HRMS(FAB) calcd for C₁₉H₂₅FN₃O₅S (M+H⁺) 426.1499, found 426.1508. Anal. calcd for C₁₉H₂₄FN₃O₅S: C, 53.63; H, 5.68; N, 9.88. Found: C, 53.69; H, 5.74; N, 9.89.

### Example 13

### N-({(5S)-3-[3-Fluoro-4-[1-(methylimino)-1-oxidohexahydro-1λ⁴-thiopyran-4-yl]phenyl]-2-oxo-1,3-oxazolidin-5-yl}methyl)propanethioamide, Z-isomer (21)

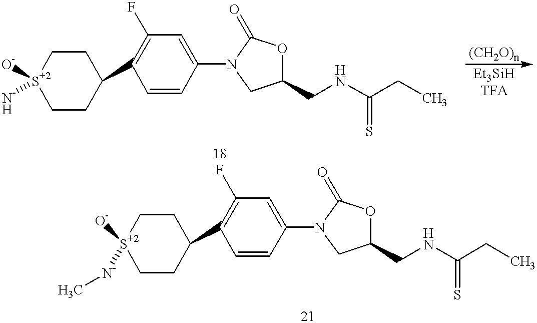

A stirred suspension of 18 (Example 10) (50 mg, 0.12 mmol) and paraformaldehyde (11 mg, 0.37 mmol) in acetonitrile (1 mL) is treated with triethylsilane (60 μL, 0.38 mmol) and trifluoroacetic acid (28 μL, 0.36 mmol) and kept at ambient temperature, under nitrogen, for 5 hours. It is then diluted with water, neutralized to pH 11 and extracted with 5% MeOH—CH₂Cl₂. The extracts are dried (Na₂SO₄) and concentrated. The residue, combined with the product of a second 0.30 mmol reaction, is chromatographed on silica gel with 3% MeOH—CHCl₃. Crystallization of the product from MeOH gives 130 mg of 21.

HRMS(FAB) calcd for C₁₉H₂₇FN₃O₃S₂ (M+H⁺) 428.1478, found 428.1481. Anal. calcd for C₁₉H₂₆FN₃O₃S₂: C, 53.37; H, 6.13; N, 9.83. Found: C, 53.34; H, 6.15; N, 9.83.

### Example 14

### N-({(5S)-3-[3-Fluoro-4-[1-(acetylimino)-1-oxidohexahydro-1λ⁴-thiopyran-4-yl]phenyl]-2-oxo-1,3-oxazolidin-5-yl}methyl)propanethioamide, Z-isomer (22)

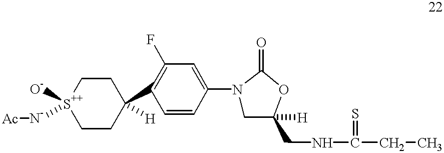

As described in Preparation 2, Compound 18 (Example 10) is allowed to react with acetic anhydride in acetic acid to give 22 which is recrystallized from MeOH.

Mp 214.0-214.5° C. (dec), HRMS(FAB) calcd for C₂₀H₂₇FN₃O₄S₂ (M+H⁺) 456.1427, found 456.1430. Anal. calcd for C₂₀H₂₆FN₃O₄S₂: C, 52.73; H, 5.75; N, 9.22. Found: C, 52.57; H, 5.76; N, 9.20.

### Example 15

### N-({(5S)-3-[3-Fluoro-4-[1-(ethylimino)-1-oxidohexahydro-1λ⁴-thiopyran-4-yl]phenyl]-2-oxo-1,3-oxazolidin-5-yl}methyl)propanethioamide, Z-isomer (23)

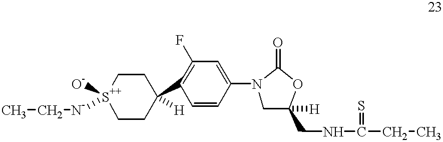

Compound 23 is prepared according to the procedure described in Example 13 by substituting acetaldehyde for paraformaldehyde. It is purified by silica gel chromatography with 2% MeOH—CHCl₃ and recrystallization from MeOH.

Mp 200-201° C.; HRMS(FAB) calcd for C₂₀H₂₉FN₃O₃S₂ (M+H⁺) 442.1634, found 442.1645.

### Example 16

### N-({(5S)-3-[3-Fluoro-4-[1-[(phenylmethyl)imino]-1-oxidohexahydro-1λ⁴-thiopyran-4-yl]phenyl]-2-oxo-1,3-oxazolidin-5-yl}methyl)propanethioamide, Z-isomer (24)

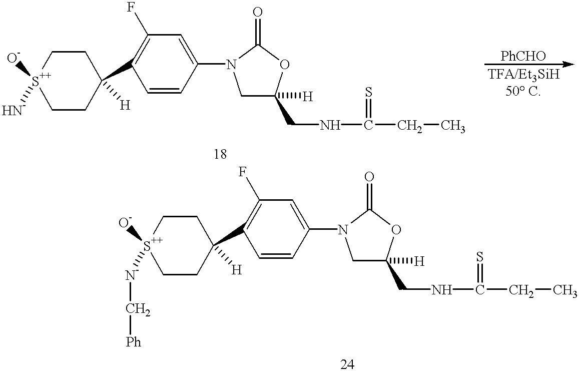

A stirred suspension of 18 (Example 10) (151 mg, 0.37 mmol) in acetonitrile (3 mL) is treated with benzaldehyde (115 μL, 1.13 mmol), trifluoroacetic acid (85 μL, 1.10 mmol) and triethylsilane (175 μL, 1.10 mmol) and kept at 50° C., under nitrogen, for 20 hours. It is then mixed with water, neutralized to pH 11 and extracted with 5% MeOH—CH₂Cl₂. The extract is dried (NaSO₄) and concentrated. Chromatography of the residue on silica gel first with 2% MeOH—CHCl₃ and then with 15% acetone-1% MeOH—CHCl₃ and crystallization of the resulting product from MeOH gives 24.

Mp 207-208° C.; HRMS(FAB) calcd for C₂₅H₃₁FN₃O₃S₂ (M+H⁺) 504.1790, found 504.1796. Anal. calcd for C₂₅H₃₀FN₃O₃S₂: C, 59.62; H, 6.00; N, 8.34. Found: C, 59.55; H, 6.03; N, 8.33.

### Example 17

### N-({(5S)-3-[3-Fluoro-4-[1-[(3-phenylpropyl)imino]-1-oxidohexahydro-1λ⁴-thiopyran-4-yl]phenyl]-2-oxo-1,3-oxazolidin-5-yl}methyl)propanethioamide, Z-isomer (25)

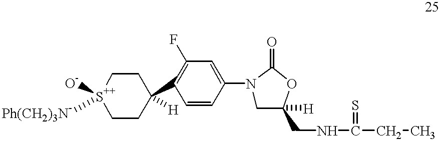

Compound 25 is prepared by the procedure described in Example 16 by substituting 3-phenylpropionaldehyde for benzaldehyde.

Mp 165.5-167° C.; HRMS(FAB) calcd for C₂₇H₃₅FN₃O₃S₂ (M+H⁺) 532.2104, found 532.2114. Anal. calcd for C₂₇H₃₄FN₃O₃S₂: C, 60.99; H, 6.45; N, 7.90. Found: 60.65; H, 6.53; N, 7.78.

### Example 18

### N-({(5S)-3-[3-Fluoro-4-(1-{[(methylamino)carbonyl]imino}-1-oxidohexahydro-1λ⁴-thiopyran-4-yl)phenyl]-2-oxo-1,3-oxazolidin-5-yl}methyl)propanethioamide, Z-isomer (26)

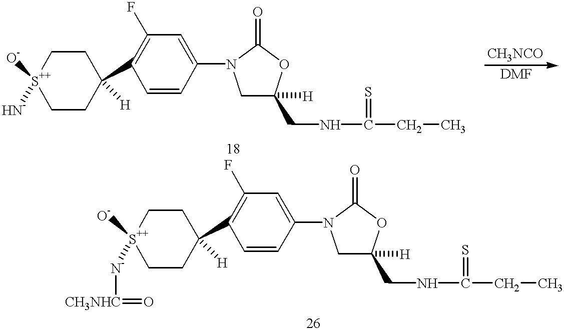

A stirred solution of 18 (Example 10) (152 mg, 0.37 mmol) in dimethylformamide (3 mL), under nitrogen, is treated with methylisocyanate (24 μL, 0.41 mmol) and kept at ambient temperature (24° C.) or 67 hours. It is concentrated in vacuo and the residue is chromatographed on silica gel with 30% acetone-1% MeOH—CHCl₃. Crystallization of the product from MeOH gives 133 mg of 26.

Mp 203-204° C.; HRMS(FAB) calcd for C₂₀H₂₈FN₄O₄S₂ (M+H⁺) 471.1536, found 471.1538. Anal. calcd for C₂₀H₂₇FN₄O₄S₂: C, 51.05; H, 5.78; N, 11.91. Found: C, 51.01; H, 5.83; N, 11.88.

### Example 19

### N-({(5S)-3-[3-Fluoro-4-(1-[(methoxycarbonyl)imino]-1-oxidohexahydro-1λ⁴-thiopyran-4-yl)phenyl]-2-oxo-1,3-oxazolidin-5-yl}methyl)propanethioamide, Z-isomer (27)

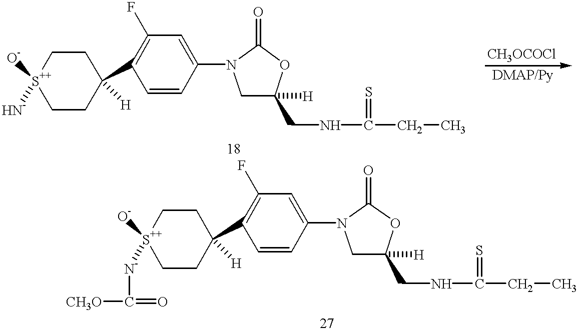

A stirred solution of 18 (Example 10) (151 mg, 0.365 mmol) and 4-(dimethylamino)pyridine (5.3 mg, 0.043 mol) in pyridine (3 mL), under nitrogen, is treated with methyl chloroformate (56 μL, 0.72 mmol) and kept at ambient temperature (24° C.) for 5 hours. Additional methyl chloroformate (56 μL) is added and the mixture is kept at ambient temperature for 2 hours and concentrated in vacuo. Chromatography of the residue on silica gel with 2% MeOH—CHCl₃ and crystallization of the product from acetonitrile-MeOH gives 132 mg of 27.

Mp 217-218° C.; HRMS(FAB) calcd for C₂₀H₂₇FN₃O₅S₂ (M+H⁺) 472.1376, found 472.1385. Anal. calcd for C₂₀H₂₆FN₃O₅S₂: C, 50.92; H, 5.56; N, 8.91. Found: C, 51.02; H, 5.59; N, 8.90.

### Example 20

### N-({(5S)-3-[3-Fluoro4-(1-[[(ethoxycarbonyl)methyl]imino]-1-oxidohexahydro-1λ⁴-thiopyran-4-yl)phenyl]-2-oxo-1,3-oxazolidin-5-yl}methyl)propanethioamide, Z-isomer (28)

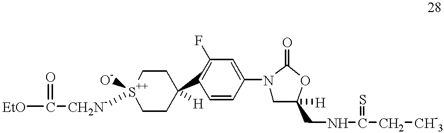

Compound 28 is prepared by the procedure described in Example 16 by substituting ethyl glyoxalate for benzaldehyde. It is purified by silica gel chromatography with 20% acetone-1% MeOH—CHCl₃ and crystallization from MeOH.

Mp 183.5-184.5° C.; HRMS(FAB) calcd for C₂₂H₃₁FN₃O₅S₂ (M+H⁺) 500.1689, found 500.1699. Anal. calcd for C₂₀H₃₀FN₃O₅S₂: C, 52.89; H, 6.05; N, 8.41. Found: C, 52.76; H, 6.04; N, 8.39.

### Example 21

### N-({(5S)-3-[3-Fluoro-4-(1-{[[(4-nitrophenyl)amino]carbonyl]imino}-1-oxidohexahydro-1λ⁴-thiopyran-4-yl)phenyl]-2-oxo-1,3-oxazolidin-5-yl}methyl)propanethioamide, Z-isomer (29)

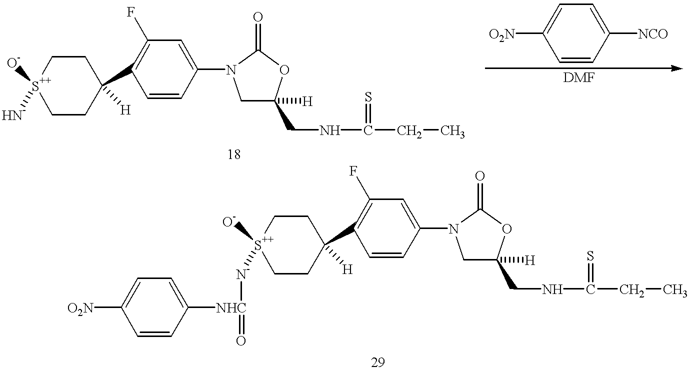

A stirred mixture of 18 (Example 10) (151 mg, 0.37 mmol), 4-nitrophenylisocyanate (79 mg, 0.48 mmol) and dimethylformamide (3 mL) is kept, under nitrogen, for 18 hours and concentrated in vacuo. Chromatography of the residue on silica gel first with 4% MeOH—CHCl₃ and then with 12.5% acetone-1% MeOH—CHCl₃ gives the product which is triturated with MeOH—CH₂Cl₂ to give 166 mg of 29.

Mp 222-228° C.; HRMS(FAB) calcd for C₂₅H₂₉FN₅O₆S₂ (M+H⁺) 578.1543, found 578.1534. Anal. calcd for C₂₅H₂₈FN₅O₆S₂; C, 51.98; H, 4.89; N, 12.12. Found: C, 51.83; H, 4.91; N, 12.01.

### Example 22

### N-({(5S)-3-[3-Fluoro-4-[1-[(aminocarbonyl)imino]-1-oxidohexahydro-1λ⁴-thiopyran-4-yl]phenyl]-2-oxo-1,3-oxazolidin-5-yl}methyl)propanethioamide, Z-isomer (30)

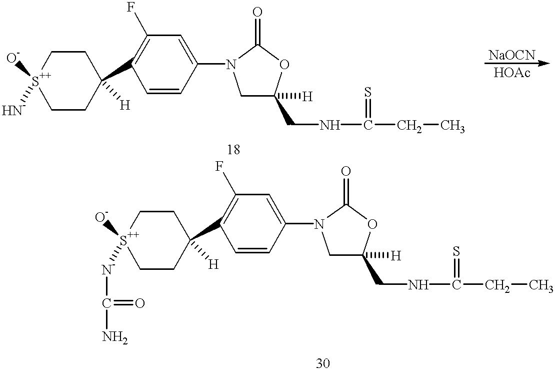

A stirred solution of 18 (Example 10) (151 mg, 0.365 mmol) in acetic acid (5 mL) is treated with sodium isocyanate (245 mg, 3.77 mmol) and kept, under nitrogen, at ambient temperature for 19 hours. It is then concentrated in vacuo. A mixture of the residue in water and 5% MeOH—CH₂Cl₂ is neutralized to pH5 with 1 N NaOH and then concentrated in vacuo. A mixture of the residue, MeOH and silica gel is concentrated and the residue is extracted with 5% MeOH—CHCl₃. The extract is concentrated and the residue is chromatographed on silica gel first with 5% MeOH—CHCl₃ and then with 4% MeOH—CHCl₃. Crystallization of the product from MeOH—CHCl₃ gives 50 mg of 30.

Mp 236-238° C. (dec); HRMS(FAB) calcd for C₁₉H₂₆FN₄O₄S₂ (M+H⁺) 457.1379, found 457, 1382. Anal. calcd for C₁₉H₂₅FN₄O₄S₂: C, 49.98; H, 5.52; N, 12.27. Found: C, 49.65; H, 5.61; N, 12.05.

### Example 23

### N-({(5S)-3-[3-Fluoro-4-[1-[[(aminocarbonyl)methyl]imino]-1-oxidohexahydro-1λ⁴-thiopyran-4-yl]phenyl]-2-oxo-1,3-oxazolidin-5-yl}methyl)propanethioamide, Z-isomer (31)

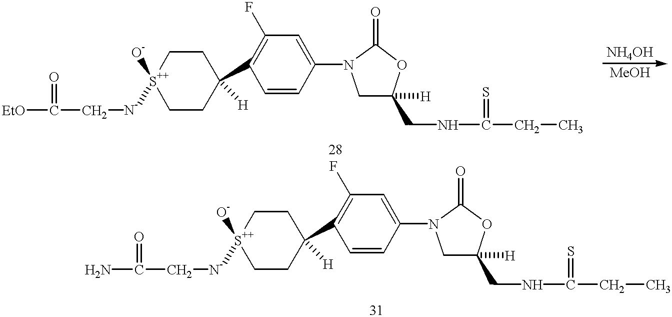

A stirred suspension of 28 (Example 20) (161 mg, 0.322 mmol) in MeOH (13 mL) is treated with 28% ammonium hydroxide (3.2 mL), kept at ambient temperature for 65 hours and concentrated in vacuo. Chromatography of the residue on silica gel with 6% MeOH—CHCl₃ and crystallization of the product from MeOH gives 98 mg of 31.

Mp 221-222° C.; HRMS(FAB) calcd for C₂₀H₂₈FN₄O₄S₂ (M+H⁺) 471.1536, found 471.1540. Anal. calcd for C₂₀H₂₇FN₄O₄S₂: C, 51.05; H, 5.78; N, 11.91. Found: C, 51.02; H, 5.80; N, 11.90.

### Example 24

N-({(5S)-3-[3-Fluoro-4-[1-[(2-hydroxyethyl)imino]-1-oxidohexahydro-1λ⁴-thiopyran-4-yl]phenyl]-2-oxo-1,3-oxazolidin-5-yl}methyl)propanethioamide, Z-isomer (32)

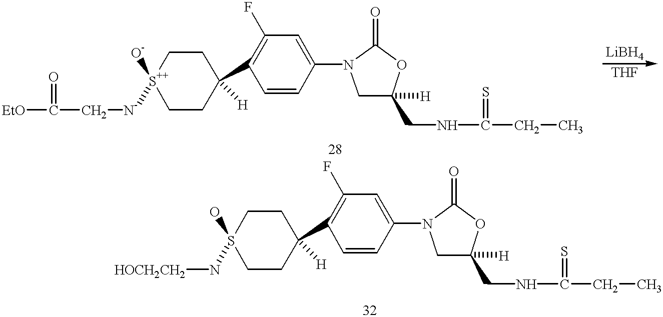

A stirred solution of 28 (Example 20) (240 mg, 0.48 mmol) in THF (5 mL) is treated with a 2.0 M solution of lithium borohydride in THF (0.24 mL, 0.48 mmol) and kept, under nitrogen, at ambient temperature for 4 hours. It is then mixed with a little water, treated, dropwise with enough 10% aqueous NaHSO₄ to give pH 2, stirred for 5 minutes and poured into saturated aqueous NaHCO₃. The pH is raised to 10 with 1 N NaOH and the mixture is extracted with 5% MeOH—CH₂Cl₂. The extract is dried (NaSO₄) and concentrated. Chromatography of the residue on silica gel with 5% MeOH—CH₂Cl₂ and crystallization of the product from MeOH gives 73 mg of 32.

Mp 180-181° C. (dec); HRMS(FAB) calcd for C₂₀H₂₉FN₃O₄S₂ (M+H⁺) 458.1583, found 458.1580. Anal. calcd for C₂₀H₂₈FN₃O₄S₂: C, 52.50; H, 6.17; N, 9.18. Found: C, 52.64; H, 6.34, N, 8.98.

### Example 25

### N-[((5S)-3-{3-Fluoro-4-[1-(methylimino)-1-oxido-1λ⁴,4-thiazinan-4-yl]phenyl}-2-oxo-1,3-oxazolidin-5-yl)methyl]propanethioamide (33)

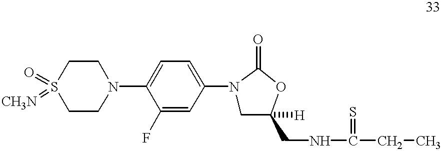

Compound 33 is prepared by the procedure described in Example 13 by substituting compound 5 (Example 2) for compound 18. It is purified by silica gel chromatography first with 20% acetone-1% MeOH—CHCl₃ and then with 4% MeOH—CHCl₃. HRMS(FAB) calcd for C₁₈H₂₆FN₄O₃S₂ (M+H⁺) 429.1430, found 429.1436.

### Example 26

### N-[((5S)-3-{3-Fluoro-4-[1-(methylimino)-1-oxido-1λ⁴,4-thiazinan-4-yl]phenyl}-2-oxo-1,3-oxazolidin-5-yl)methyl]cyclopropanecarbothioamide (34)

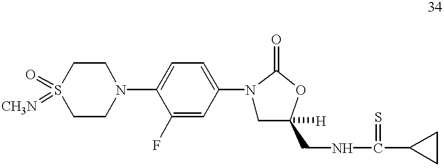

Compound 34 is prepared by the procedure described in Example 13 by substituting compound 6 (Example 3) for compound 18. It is purified by silica gel chromatography with 3% MeOH—CH₂Cl₂.

HRMS(FAB) calcd for C₁₉H₂₆FN₄O₃S₂ (M+H⁺) 441.1430, found 441.1425. Anal. calcd for C₁₉H₂₅FN₄O₃S₂: C, 51.80, H, 5.72; N, 12.72. Found: C, 51.60; H, 6.03; N, 12.34.

### Example 27

### N-[((5S)-3-{3-Fluoro-4-(1-[(methoxycarbonyl)imino]-1-oxido-1λ⁴,4-thiazinan-4-yl)phenyl}-2-oxo-1,3-oxazolidin-5-yl)methyl]propanethioamide (35)

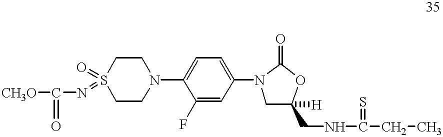

Compound 35 is prepared by the procedure described in Example 19 by substituting compound 5 (Example 2) for compound 18. It is purified by silica gel chromatography with 3% MeOH—CHCl₃ and crystallization from acetonitrile-MeOH.

Mp 211-212° C. (dec); HRMS(FAB) calcd for C₁₉H₂₆FN₄O₅S₂ (M+H⁺) 473.1328, found 473.1329. Anal. calcd for C₁₉H₂₅FN₄O₅S₂: C, 48.29; H, 5.33; N, 11.86. Found: C, 48.34; H, 5.41; N, 11.87.

### Example 28

### N-[((5S)-3-{3-Fluoro-4-(1-[(methoxycarbonyl)imino]-1-oxido-1λ⁴,4-thiazinan-4-yl)phenyl}-2-oxo-1,3-oxazolidin-5-yl)methyl]cyclopropanecarbothioamide (36)

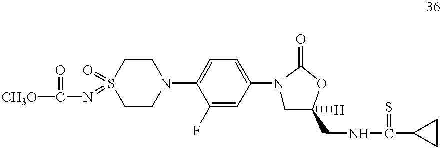

Compound 36 is prepared by the procedure described in Example 19 by substituting compound 6 (Example 3) for compound 18. It is purified by silica gel chromatography first with 2.5% MeOH—CHCl₃ and then with 10% acetone-CHCl₃ and crystallization from acetonitrile-MeOH.

Mp 208-209° C. (dec). Anal. calcd for C₂₀H₂₅FN₄O₅S₂; C, 49.57; H, 5.20; N, 11.56. Found: C, 49.55; H, 5.22; N, 11.58.

### Example 29

N-({(5S)-3-[3-Fluoro-4-[1-(methylimino)-1-oxidohexahydro-1λ⁴-thiopyran-4-yl]phenyl]-2-oxo-1,3-oxazolidin-5-yl}methyl)cyclopropanecarbothioamide, Z-isomer (37)

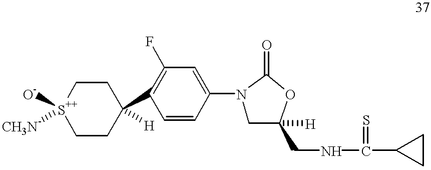

Compound 37 is prepared by the procedure described in Example 13 by substituting compound 19 (Example 11) for compound 18. It is purified by crystallization from MeOH—CH₂Cl₂.

Mp 201-202° C. (dec); HRMS(FAB) calcd for C₂₀H₂₇FN₃O₃S₂ (M+H⁺) 440.1478, found 440.1475. Anal. calcd for C₂₀H₂₆FN₃O₃S₂: C, 54.65; H, 5.96; N, 9.56. Found: C, 54.12; H, 6.16; N, 9.44.

### Example 30

### N-[((5S)-3-{3-Fluoro-4-[1-[(methoxycarbonyl)imino]-1-oxidohexahydro-1λ⁴-thiopyran-4-yl]phenyl}-2-oxo-1,3-oxazolidin-5-yl)methyl]cyclopropanecarbothioamide, Z-isomer (38)

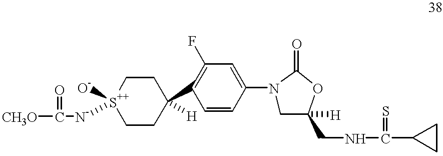

Compound 38 is prepared according to the procedure described in Example 19 by substituting compound 19 (Example 11) for compound 18. It is purified by silica gel chromatography with 7.5% acetone-1% MeOH—CHCl₃ and crystallization from MeOH—CH₂Cl₂.

Mp 219-220° C. (dec); HRMS(FAB) calcd for C₂₁H₂₇FN₃O₅S₂ (M+H⁺) 484.1376, found 484.1389. Anal. calcd for C₂₁H₂₆FN₃O₅S₂: C, 52.16; H, 5.42; N, 8.69. Found: C, 52.35; H, 5.50; N, 8.58.

### Example 31

### N-[((5S)-3-{3-Fluoro-4-[1-(methylimino)-1-oxidohexahydro-1λ⁴-thiopyran-4-yl]phenyl}-2-oxo-1,3-oxazolidin-5-yl)methyl]cyclopropanecarbothioamide, E-isomer (39)

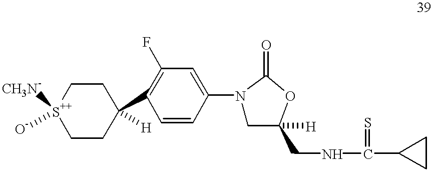

Compound 39 is prepared by the procedure described in Example 13 by substituting compound 14 (Example 7) for compound 18. It is purified by silica gel chromatography first with 3% MeOH—CHCl₃ and then with 1% MeOH—EtOAc.

HRMS(FAB) calcd for C₂₀H₂₇FN₃O₃S₂ (M+H⁺) 440.1478, found 440.1473.

### Example 32

### N-[((5S)-3-{3-Fluoro-4-[1-(methylimino)-1-oxidohexahydro-1λ⁴-thiopyran-4-yl]phenyl}-2-oxo-1,3-oxazolidin-5-yl)methyl]propanethioamide, E-isomer (40)

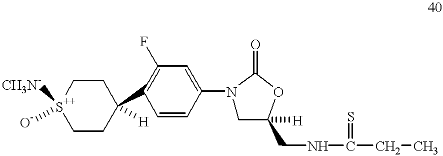

Compound 40 is prepared by the procedure described in Example 13 by substituting compound 13 (Example 6) for compound 18. It is purified by silica gel chromatography with 1% MeOH—EtOAc.

HRMS(FAB) calcd for C₁₉H₂₇FN₃O₃S₂ (M+H⁺) 428.1478, found 428.1484.

### Example 33

### N-[((5S)-3-{3-Fluoro-4-[1-[[(phenylmethoxy)carbnonyl]imino]-1-oxidohexahydro-1λ⁴-thiopyran-4-yl]phenyl}-2-oxo-1,3-oxazolidin-5-yl)methyl]acetamide, Z-isomer (41)

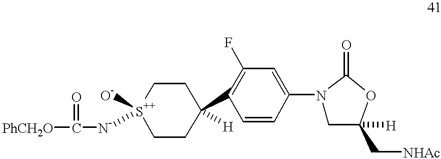

Compound 41 is prepared according to the procedure described in Example 19 by substituting compound 15 (Example 8) for compound 18 and benzyl chloroformate for methyl chloroformate. It is purified by silica gel chromatography with 3% MeOH—CHCl₃ and recrystallization from MeOH.

Mp 213-214° C. (dec); HRMS(FAB) calcd for C₂₅H₂₉FN₃O₆S (M+H⁺) 518.1761, found 518.1763. Anal. calcd for C₂₅H₂₈FN₃O₆S: C, 58.01; H, 5.45; N, 8.12. Found: C, 57.91; H, 5.63; N, 8.11.

### Example 34

### N-({(5S)-3-[3-Fluoro-4-(1-{[(benzylamino)carbonyl]imino}-1-oxidohexahydro-1λ⁴-thiopyran-4-yl)phenyl]-2-oxo-1,3-oxazolidin-5-yl}methyl)acetamide, Z-isomer (42)

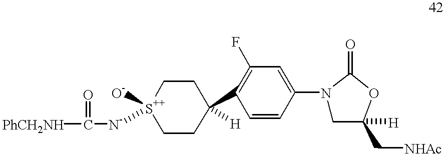

Compound 42 is prepared according to the procedure described in Example 18 by substituting compound 15 (Example 8) for compound 18 and benzylisocyanate for methylisocyanate. It is purified by crystallization from MeOH.

Mp 238.5-239.5° C. (dec); HRMS(FAB) calcd for C₂₅H₃₀FN₄O₅S (M+H⁺) 517.1921, found 517.1927. Anal. calcd for C₂₅H₂₉FN₄O₅S: C, 58.13; H, 5.66; N, 10.85. Found: C, 57.96; H, 5.80; N, 10.90.

1. A compound of formula I 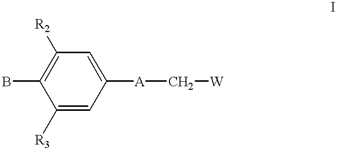
or a pharmaceutically acceptable salt thereof wherein:
A is a structure i, ii, iii, or iv 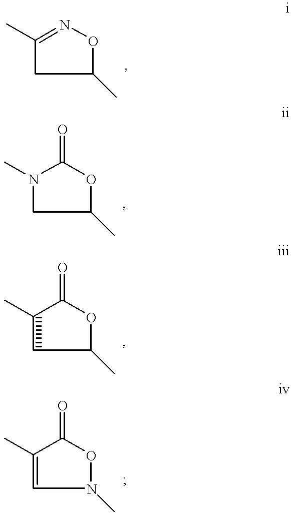
B is 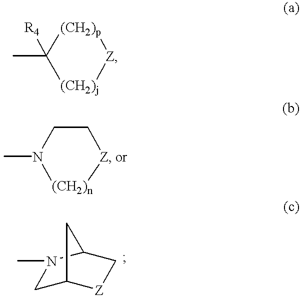
W is NHC(═X)R₁, or -Y-het; povided that when A is a structure iv, W is not -Y-het;
X is O, or S; provided that when X is O, B is not the subsection (b).
Y is NH, O, or S;
Z is S(═O)(═N—R₅);
R₁ is
(a) H,
(b) NH₂,
(c) NHC₁₋₄alkyl,
(d) C₁₋₄alkyl,
(e) C₂₋₄alkenyl,
(f) OC₁₋₄alkyl,
(g) SC₁₋₄alkyl, or
(h) (CH₂)ₚC₃₋₆cycloalkyl;
at each occurrence, alkyl or cycloalkyl in R₁ is optionally substituted with one or more F, Cl or CN;
R₂ and R₃ are independently H, F, Cl, methyl or ethyl;
R₄ is H, CH₃, or F;
R₅ is
(a) H,
(b) C₁₋₄alkyl,
(c) C(═O)C₁₋₄alkyl,
(d) C(═O)OC₁₋₄alkyl,
(e) C(═O)NHR₆, or
(f) C(═S)NHR₆;
R₆ is H, C₁₋₄alkyl, or phenyl;
at each occurrence, alkyl in R₅ and R₆ is optionally substituted with one or more halo, CN, NO₂, phenyl, C₃₋₆ cycloalkyl, OR₇, C(═O)R⁷, OC(═O)R₇, C(═O)OR₇, S(═O)ₘR₇, S(═O)ₘNR₇R₇, NR₇SO₂R₇, NR₇SO₂NR₇R₇, NR₇C(═O)R₇, C(═O)NR₇R₇, NR₇R₇, oxo, or oxime;
R₇ is H, C₁₋₄alkyl, or phenyl;
at each occurrence, phenyl is optionally substituted with one or more halo, CN, NO₂, phenyl, C₃₋₆cycloalkyl, OR₇, C(═O)R⁷, OC(═O)R₇, C(═O)OR₇, S(═O)ₘR₇, S(═O)ₘNR₇R₇, NR₇SO₂R₇, NR₇SO₂NR₇R₇, NR₇C(═O)R₇, C(═O)NR₇R₇, or NR₇R₇;
het is a C-linked five-(5) membered heteroaryl ring having 1-4 heteroatoms selected from the group consisting of oxygen, sulfur, and nitrogen, or het is a C-linked six (6) membered heteroaryl ring having 1-3 nitrogen atoms;
p is 0, 1, or 2;
j is 1, 2, 3, 4, or 5; provided that k and j taken together are 2, 3, 4 or 5;
m is 0, 1, or 2;
n is 2 or 3; and [symbol] in structure iii is either a double bond or a single bond.

2. A compound of formula I which is a compound of formula IA:

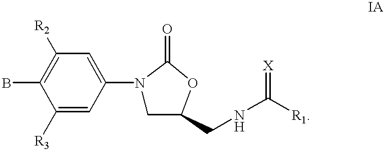

3. A compound of claim 2 wherein R₁ is C₁₋₄alkyl.

4. A compound of claim 2 wherein R₁ is ethyl.

5. A compound of claim 2 wherein R₁ is methyl.

6. A compound of claim 2 wherein R₁ is C₃₋₆cycloalkyl.

7. A compound of claim 2 wherein R₁ is cyclopropyl.

8. A compound of claim 2-7 wherein X is sulfur atom.

9. A compound of claim 2-7 wherein X oxygen atom.

10. A compound of claim 8 wherein one of R₂ and R₃ is H, the other one is F.

11. A compound of claim 9 wherein one of R₂ and R₃ is H, the other one is F.

12. A compound of claim 8 wherein R₄ is H.

13. A compound of claim 9 wherein R₄ is H.

14. A compound of claim 8 wherein structure B is 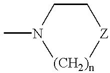
wherein Z is S(═O)(═NR₅).

15. A compound of claim 9 wherein structure B is 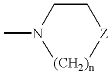
wherein Z is S(═O)(═NR₅).

16. A compound of claim 8 wherein structure B is 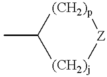
wherein Z is S(═O)(═NR₅)

17. A compound of claim 8 wherein structure B is 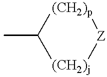
wherein Z is S(═O)(═NR₅).

18. A compound of claim 14-17 wherein R₅ is H.

19. A compound of claim 14-17 wherein R₅ is C₁₋₄alkyl, optionally substituted with OH; or C₁₋₄alkyl substituted with C(═O)NHC₁₋₄alkyl, C(═O)NH₂ or phenyl; wherein the phenyl is optionally substituted with OH, methyl, NO₂, CF₃, or CN.

20. A compound of claim 20 wherein R₅ is CH₃, or ethyl.

21. A compound of claim 20 wherein R₅ is C₁₋₄alkyl substituted with phenyl wherein the phenyl is optionally substituted with NO₂.

22. A compound of claim 14-17 wherein R₅ is C(═O)C₁₋₄alkyl, C(═O)OC₁₋₄alkyl, C(═O)NH₂, or C(═O)NHC₁₋₄alkyl.

23. A compound of claim 22 wherein R₅ is C(═O)NHCH₃, or C(═O)NHCH₂CH₃.

24. A compound of claim 14-17 wherein R₅ is C(═O)CH₃.

25. A compound of claim 14-17 wherein R₅ is C(═O)OCH₃.

26. A compound of claim 2 which is
(1) N-({(5S)-3-[3-fluoro-4-(1-imino-1-oxido-1λ⁴,4-thiazinan-4-yl)phenyl]-2-oxo-1,3-oxazolidin-5-yl}methyl)ethanethioamide;
(2) N-({(5S)-3-[3-fluoro-4-(1-imino-1-oxido-1λ⁴,4-thiazinan-4-yl)phenyl]-2-oxo-1,3-oxazolidin-5-yl}methyl)propanethioamide;
(3) N-({(5S)-3-[3-fluoro-4-(1-imino-1-oxido-1λ⁴,4-thiazinan-4-yl)phenyl]-2-oxo-1,3-oxazolidin-5-yl}methyl)cyclopropanecarbothioamide;
(4) N-({(5S)-3-[3-fluoro-4-(1-imino-1-oxidohexahydro-1λ⁴-thiopyran-4-yl)phenyl]-2-oxo-1,3-oxazolidin-5-yl}methyl)acetamide (E)-isomer;
(5) N-({(5S)-3-[3-fluoro-4-(1-imino-1-oxidohexahydro-1λ⁴-thiopyran-4-yl)phenyl]-2-oxo-1,3-oxazolidin-5-yl}methyl)ethanethioamide (E)-isomer;
(6) N-({(5S)-3-[3-fluoro-4-(1-imino-1-oxidohexahydro-1λ⁴-thiopyran-4-yl)phenyl]-2-oxo-1,3-oxazolidin-5-yl}methyl)propanethioamide (E)-isomer;
(7) N-({(5S)-3-[3-fluoro-4-(1-imino-1-oxidohexahydro-1λ⁴-thiopyran-4-yl)phenyl]-2-oxo-1,3-oxazolidin-5-yl}methyl)cyclopropanecarbothioamide (E)-isomer;
(8) N-({(5S)-3-[3-fluoro-4-(1-imino-1-oxidohexahydro-1λ⁴-thiopyran-4-yl)phenyl]-2-oxo-1,3-oxazolidin-5-yl}methyl)acetamide (Z)-isomer;
(9) N-({(5S)-3-[3-fluoro-4-(1-imino-1-oxidohexahydro-1λ⁴-thiopyran-4-yl)phenyl]-2-oxo-1,3-oxazolidin-5-yl}methyl)ethanethioamide (Z)-isomer;
(10) N-({(5S)-3-[3-fluoro-4-(1-imino-1-oxidohexahydro-1λ⁴-thiopyran-4-yl)phenyl]-2-oxo-1,3-oxazolidin-5-yl}methyl)propanethioamide (Z)-isomer;
(11) N-({(5S)-3-[3-fluoro-4-(1-imino-1-oxidohexahydro-1λ⁴-thiopyran-4-yl)phenyl]-2-oxo-1,3-oxazolidin-5-yl}methyl)cyclopropanethioamide (Z)-isomer;
(12) N-({(5S)-3-[3-fluoro-4-[1-(acetylimino)-1-oxidohexahydro-1λ⁴-thiopyran-4-yl]phenyl]-2-oxo-1,3-oxazolidin-5-yl}methyl)acetamide, Z-isomer;
(13) N-({(5S)-3-[3-fluoro-4-[1-(methylimino)-1-oxidohexahydro-1λ⁴-thiopyran-4-yl]phenyl]-2-oxo-1,3-oxazolidin-5-yl}methyl)propanethioamide, Z-isomer;
(14) N-({(5S)-3-[3-fluoro-4-[1-(acetylimino)-1-oxidohexahydro-1λ⁴-thiopyran-4-yl]phenyl]-2-oxo-1,3-oxazolidin-5-yl}methyl)propanethioamide, Z-isomer;
(15) N-({(5S)-3-[3-fluoro-4-[1-(ethylimino)-1-oxidohexahydro-1λ⁴-thiopyran-4-yl]phenyl]-2-oxo-1,3-oxazolidin-5-yl}methyl)propanethioamide, Z-isomer;
(16) N-({(5S)-3-[3-fluoro-4-[1-[(phenylmethyl)imino]-1-oxidohexahydro-1λ⁴-thiopyran-4-yl]phenyl]-2-oxo-1,3-oxazolidin-5-yl}methyl)propanethioamide, Z-isomer;
(17) N-({(5S)-3-[3-fluoro-4-[1-[(3-phenylpropyl)imino]-1-oxidohexahydro-1λ⁴-thiopyran-4-yl]phenyl]-2-oxo-1,3-oxazolidin-5-yl}methyl)propanethioamide, Z-isomer;
(18) N-({(5S)-3-[3-fluoro-4-(1-{[(methylamino)carbonyl]imino}-1-oxidohexahydro-1λ⁴-thiopyran-4-yl)phenyl]-2-oxo-1,3-oxazolidin-5-yl}methyl)propanethioamide, Z-isomer;
(19) N-({(5S)-3-[3-fluoro-4-(1-[(methoxycarbonyl)imino]-1-oxidohexahydro-1λ⁴-thiopyran-4-yl)phenyl]-2-oxo-1,3-oxazolidin-5-yl}methyl)propanethioamide, Z-isomer;
(20) N-({(5S)-3-[3-fluoro-4-(1-[[(ethoxycarbonyl)methyl]imino]-1-oxidohexahydro-1λ⁴-thiopyran-4-yl)phenyl]-2-oxo-1,3-oxazolidin-5-yl}methyl)propanethioamide, Z-isomer;
(21) N-({(5S)-3-[3-fluoro-4-(1-{[[(4-nitrophenyl)amino]carbonyl]imino}-1-oxidohexahydro-1λ⁴-thiopyran-4-yl)phenyl]-2-oxo-1,3-oxazolidin-5-yl}methyl)propanethioamide, Z-isomer;
(22) N-({(5S)-3-[3-fluoro-4-[1-[(aminocarbonyl)imino]-1-oxidohexahydro-1λ⁴-thiopyran-4-yl]phenyl]-2-oxo-1,3-oxazolidin-5-yl}methyl)propanethioamide, Z-isomer;
(23) N-({(5S)-3-[3-fluoro-4-[1-[[(aminocarbonyl)methyl]imino]-1-oxidohexahydro-1λ⁴-thiopyran-4-yl]phenyl]-2-oxo-1,3-oxazolidin-5-yl}methyl)propanethioamide, Z-isomer;
(24) N-({(5S)-3-[3-fluoro4-[1-[(2-hydroxyethyl)imino]-1-oxidohexahydro-1λ⁴-thiopyran-4-yl]phenyl]-2-oxo-1,3-oxazolidin-5-yl}methyl)propanethioamide, Z-isomer;
(25) N-[((5S)-3-{3-fluoro-4-[1-(methylimino)-1-oxido-1λ⁴,4-thiazinan-4-yl]phenyl}-2-oxo-1,3-oxazolidin-5-yl)methyl]propanethioamide;
(26) N-[((5S)-3-{3-fluoro-4-[1-(methylimino)-1-oxido-1λ⁴,4-thiazinan-4-yl]phenyl}-2-oxo-1,3-oxazolidin-5-yl)methyl]cyclopropanecarbothioamide;
(27) N-[((5S)-3-{3-fluoro-4-(1-[(methoxycarbonyl)imino]-1-oxido-1λ⁴,4-thiazinan-4-yl)phenyl}-2-oxo-1,3-oxazolidin-5-yl)methyl]propanethioamide;
(28) N-[((5S)-3-{3-fluoro-4-(1-[(methoxycarbonyl)imino]-1-oxido-1λ⁴,4-thiazinan-4-yl)phenyl}-2-oxo-1,3-oxazolidin-5-yl)methyl]cyclopropanecarbothioamide;
(29) N-({(5S)-3-[3-fluoro-4-[1-(methylimino)-1-oxidohexahydro-1λ⁴-thiopyran-4-yl]phenyl]-2-oxo-1,3-oxazolidin-5-yl}methyl)cyclopropanecarbothioamide, Z-isomer;
(30) N-[((5S)-3-{3-fluoro-4-[1-[(methoxycarbonyl)imino]-1-oxidohexahydro-1λ⁴-thiopyran-4-yl]phenyl}-2-oxo-1,3-oxazolidin-5-yl)methyl]cyclopropanecarbothioamide, Z-isomer;
(31) N-[((5S)-3-{3-fluoro-4-[1-(methylimino)-1-oxidohexahydro-1λ⁴-thiopyran-4-yl]phenyl}-2-oxo-1,3-oxazolidin-5-yl)methyl]cyclopropanecarbothioamide, E-isomer;
(32) N-[((5S)-3-{3-fluoro-4-[1-(methylimino)-1-oxidohexahydro-1λ⁴-thiopyran-4-yl]phenyl}-2-oxo-1,3-oxazolidin-5-yl)methyl]propanethioamide, E-isomer;
(33) N-[((5S)-3-{3-fluoro-4-[1-[[(phenylmethoxy)carbnonyl]imino]-1-oxidohexahydro-1λ⁴-thiopyran-4-yl]phenyl}-2-oxo-1,3-oxazolidin-5-yl)methyl]acetamide, Z-isomer; or
(34) N-({(5S)-3-[3-Fluoro-4-(1-{[(benzylamino)carbonyl]imino}-1-oxidohexahydro-1λ⁴-thiopyran-4-yl)phenyl]-2-oxo-1,3-oxazolidin-5-yl}methyl)acetamide, Z-isomer.

27. A compound of claim 2 which is
(1) N-({(5S)-3-[3-fluoro-4-(1-imino-1-oxido-1λ⁴,4-thiazinan-4-yl)phenyl]-2-oxo-1,3-oxazolidin-5-yl}methyl)ethanethioamide;
(2) N-({(5S)-3-[3-fluoro-4-(1-imino-1-oxido-1λ⁴,4-thiazinan-4-yl)phenyl]-2-oxo-1,3-oxazolidin-5-yl}methyl)propanethioamide;
(3) N-({(5S)-3-[3-fluoro-4-(1-imino-1-oxido-1λ⁴,4-thiazinan-4-yl)phenyl]-2-oxo-1,3-oxazolidin-5-yl}methyl)cyclopropanecarbothioamide;
(4) N-({(5S)-3-[3-fluoro-4-(1-imino-1-oxidohexahydro-1λ⁴-thiopyran-4-yl)phenyl]-2-oxo-1,3-oxazolidin-5-yl}methyl)ethanethioamide (Z)-isomer;
(5) N-({(5S)-3-[3-fluoro-4-(1-imino-1-oxidohexahydro-1λ⁴-thiopyran-4-yl)phenyl]-2-oxo-1,3-oxazolidin-5-yl}methyl)propanethioamide (Z)-isomer; or
(6) N-({(5S)-3-[3-fluoro-4-(1-imino-1-oxidohexahydro-1λ⁴-thiopyran-4-yl)phenyl]-2-oxo-1,3-oxazolidin-5-yl}methyl)cyclopropanethioamide (Z)-isomer.

28. A compound of claim 2 which is
(1) N-({(5S)-3-[3-fluoro-4-[1-(methylimino)-1-oxidohexahydro-1λ⁴-thiopyran-4-yl]phenyl]-2-oxo-1,3-oxazolidin-5-yl}methyl)propanethioamide, Z-isomer;
(2) N-({(5S)-3-[3-fluoro-4-[1-(acetylimino)-1-oxidohexahydro-1λ⁴-thiopyran-4-yl]phenyl]-2-oxo-1,3-oxazolidin-5-yl}methyl)propanethioamide, Z-isomer;
(3) N-({(5S)-3-[3-fluoro-4-(1-[(methoxycarbonyl)imino]-1-oxidohexahydro-1λ⁴-thiopyran-4-yl)phenyl]-2-oxo-1,3-oxazolidin-5-yl}methyl)propanethioamide, Z-isomer;
(4) N-({(5S)-3-[3-Fluoro-4-(1-{[[(4-nitrophenyl)amino]carbonyl]imino}-1-oxidohexahydro-1λ⁴-thiopyran-4-yl)phenyl]-2-oxo-1,3-oxazolidin-5-yl}methyl)propanethioamide, Z-isomer;
(5) N-({(5S)-3-[3-fluoro-4-[1-(methylimino)-1-oxidohexahydro-1λ⁴-thiopyran-4-yl]phenyl]-2-oxo-1,3-oxazolidin-5-yl}methyl)cyclopropanecarbothioamide, Z-isomer; or
(6) N-[((5S)-3-{3-fluoro-4-[1-[(methoxycarbonyl)imino]-1-oxidohexahydro-1λ⁴-thiopyran-4-yl]phenyl}-2-oxo-1,3-oxazolidin-5-yl)methyl]cyclopropanecarbothioamide, Z-isomer.

29. A compund of claim 2 which is
(1) N-({(5S)-3-[3-Fluoro-4-[1-(methylimino)-1-oxidohexahydro-1λ⁴-thiopyran-4-yl]phenyl]-2-oxo-1,3-oxazolidin-5-yl}methyl)propanethioamide, Z-isomer;
(2) N-({(5S)-3-[3-Fluoro-4-[1-(ethylimino)-1-oxidohexahydro-1λ⁴-thiopyran-4-yl]phenyl]-2-oxo-1,3-oxazolidin-5-yl}methyl)propanethioamide, Z-isomer;
(3) N-({(5S)-3-[3-Fluoro-4-(1-{[(methylamino)carbonyl]imino}-1-oxidohexahydro-1λ⁴-thiopyran-4-yl)phenyl]-2-oxo-1,3-oxazolidin-5-yl}methyl)propanethioamide, Z-isomer;
(4) N-[((5S)-3-{3-Fluoro-4-[1-(methylimino)-1-oxido-1λ⁴,4-thiazinan-4-yl]phenyl}-2-oxo-1,3-oxazolidin-5-yl)methyl]propanethioamide; or
(5) N-[((5S)-3-{3-Fluoro-4-[1-(methylimino)-1-oxido-1λ⁴,4-thiazinan-4-yl]phenyl}-2-oxo-1,3-oxazolidin-5-yl)methyl]cyclopropanecarbothioamide.

30. A method for treating microbial infections comprising: administering to a mammal in need thereof an effective amount of a compound of formula I as shown in claim 1.

31. The method of claim 30 wherein said compound of formula I is administered orally, parenterally, transdermally, or topically in a pharmaceutical composition.

32. The method of claim 30 wherein said compound is administered in an amount of from about 0.1 to about 100 mg/kg of body weight/day.

33. The method of claim 30 wherein said compound is administered in an amount of from about 1 to about 50 mg/kg of body weight/day.

34. A method for treating microbial infections of claim 30 wherein the infection is skin infection.

35. A method for treating microbial infections of claim 30 wherein the infection is eye infection.

36. A pharmaceutical composition comprising a compound of claim 1 and a pharmaceutically acceptable carrier.

37. A compound of claim 1 wherein structure i, or iii is [chemical structure]
